SEQUENCE LISTING (118 sequences)

SEQ ID NO: 1 - DNA, 100 bp, Cannabis sativa
acaagaacaagtataatatagtcgagaatgattctctgttgagttctctcaaagtgattcaactctcacattcttacccaaaaatcttctttttctacag

SEQ ID NO: 2 - DNA, 100 bp, Cannabis sativa
caactgataaccttctaaatctgtctgtatgaatccttttgacacctttatttggtcttcgttatcttgttctttcggctccacaacaacttttgtctac

SEQ ID NO: 3 - DNA, 100 bp, Cannabis sativa
gtaacactgatcaagtagatggtggtggtcgccatagaagatcattctctttggcttttttaagatattcaacatacaagtccagttcatcttcttcttc

SEQ ID NO: 4 - DNA, 100 bp, Cannabis sativa
gggagttcttcagcaatgtcaagagctgttttatggtctctagtcaatgcattaacattggtgtctggaaggagtaacaactcctttactatctgcaagg

SEQ ID NO: 5 - DNA, 100 bp, Cannabis sativa
gatctaatagcacttggacaattgctgcaggaaagtaagtgccaacatgagtttagaagttaatgtagaagtccattttatttgattaaagacacttcct

SEQ ID NO: 6 - DNA, 100 bp, Cannabis sativa
cagccaattgaaactttctgcaagtacatgttctgtatacaatatccaccacacagatcacattattccctggttaatgcacttaaaacttgtttgcatc

SEQ ID NO: 7 - DNA, 100 bp, Cannabis sativa
taaataccccatgaggggcctgaatggttggggcttgcatcaccggagcggttagagccagaggtggtattttgggagcttgaagaaggcccaccccctg

SEQ ID NO: 8 - DNA, 100 bp, Cannabis sativa
tgtgttaaatgtagaatgttttctaagaggaatggttatgttggggaacaaccttgacagggagacaccaagggatctggtaggggtggtgatggttctg

SEQ ID NO: 9 - DNA, 100 bp, Cannabis sativa
caaaggctctctttcccattgggtagggatttatttttacctggttaatgatcttactcatctgcagttgtgtaatgtaaatcacttgtctctcttcgta

SEQ ID NO: 10 - DNA, 100 bp, Cannabis sativa
ccattgcccaacctttataaatctttcaattgtacctattccacagtcaggcaaactatgtctataatatcagtttacatggatccacccacttactttc

SEQ ID NO: 11 - DNA, 100 bp, Cannabis sativa
gatggtagtgatacatcgggtacatcggcatcaatgtgttggcgctcatcacaccagggtatacttgagagtagtgccctcctcccatgtaggccccacc

SEQ ID NO: 12 - DNA, 100 bp, Cannabis sativa
gttgttgcattttaagccttttgaaatatttgtctagagtctctgatctcattttctatagtaaactaactgctttatttgtttctttgttgtgtatgta

SEQ ID NO: 13 - DNA, 100 bp, Cannabis sativa
ccttttcactttttactttttacttctctcactataaattgaaatttgaagaagctcttccttctccatagaggaccaaacccacacaagcatcattctc

SEQ ID NO: 14 - DNA, 100 bp, Cannabis sativa
atgcatagggaatcagaactcagttttagttatgttgaaagggtttagaattgagaaatctcttgggagagatctagccttcttgaaaggttagaatagg

SEQ ID NO: 15 - DNA, 100 bp, Cannabis sativa
acacctcttgcccagagagtggaagcccaagaaatattcttccctgtcgagcaaaactagcaaggagagataaatttgtctgagtagtatcccgatcctt

SEQ ID NO: 16 - DNA, 100 bp, Cannabis sativa
tgcttacaaaaatagcactgtcttctataactccaacaaagtaaagttccaaaagtagcttcagagtactgcgcttcttcatagccttttgattcctatc

SEQ ID NO: 17 - DNA, 100 bp, Cannabis sativa
tctcaacaaacaggttcggaacagtaaaaaattgatggattcattctatttataaagcaaaaataataacaagtgttataacgagatttctaagatcaat

SEQ ID NO: 18 - DNA, 100 bp, Cannabis sativa
tttctagtctttggttcttccaggtggagattatttactttgtcttcatagtggaaaatttgggattttggactcacagagttagctcacttctcctttc

SEQ ID NO: 19 - DNA, 100 bp, Cannabis sativa
tactctggtgcttcacgtgtctatttgtgctattttgatgttcatatttatagtctagcgggaagttttttagtcatttcgttcatgaagggtcaagtac

SEQ ID NO: 20 - DNA, 100 bp, Cannabis sativa
gatctcgttttgactgaggtagtcatgccctgtttatctggtattggtcttctaggcaagatcatgagcaaaaaaacatgcaaggacatccctgtaatta

SEQ ID NO: 21 - DNA, 100 bp, Cannabis sativa
aaaataatacttttttctctagtatctttttgtaatttatacttttaactaatacatttattgtgtgtgtttgtgtttgtgttttcacagtgatgtcttc

SEQ ID NO: 22 - DNA, 100 bp, Cannabis sativa
tgaggaattggccaccccaaggctttttctagttgcctagcccgcgcagtaattaagataagccttcttggagtctccgaggtaatcaaaattgcctgca

SEQ ID NO: 23 - DNA, 100 bp, Cannabis sativa
tctgctctgtgatgggatcctgtacctcctcattagtacactttccatcattcgttgtcatctcctcagatttcacctgtttcattcaagcaaatattag

SEQ ID NO: 24 - DNA, 100 bp, Cannabis sativa
cacaaacccagtttaggaaacctctcacgcaccaactcctcaaaaacgagttgatctacctgacaaaatacaacaatattcaacagaactctcatttttc

SEQ ID NO: 25 - DNA, 100 bp, Cannabis sativa
ttaaccaaggtatccaaaccaatacctggcaatatccaacagagggattatgtcttgcataggcagtaagtagacgcctcaaggcatttctaccatcctc

SEQ ID NO: 26 - DNA, 100 bp, Cannabis sativa
atgtagtcctggtcatccatccatctcaggaatttgcgtctctgttgggtgaatgcatcacaacctacaacacttggttcttcagtttggacttggctta

SEQ ID NO: 27 - DNA, 100 bp, Cannabis sativa
atagtgattcgaattgtgtgttgatttcatagaataatacatatttatatacaaagctaggagactaaatatctactaaatatctactaaatatctaatt

SEQ ID NO: 28 - DNA, 100 bp, Cannabis sativa
gagagactcgaacttcaaaccttgtggaagcaaaccatacacgtgaccatttgaactcttaggttctcctatgcttaatcactataagaatccgacaatt

SEQ ID NO: 29 - DNA, 100 bp, Cannabis sativa
ttttgcttggataatacatagatcccatattcacaaacaactagaatgaacaagggaaacacaaacatacaatttgattggatgcagcttcatctatttt

SEQ ID NO: 30 - DNA, 100 bp, Cannabis sativa
tttattctttttgctgagttttagaactcaacatgccactaaactaaattaactaatcttcagtaaacataaaagttgtggctattaacccacttggtgg

SEQ ID NO: 31 - DNA, 100 bp, Cannabis sativa
gactctttatttcatcgatgagttgattagcatctctgagattacctgaagacaaatacctacagtaacataattattgtaacaggcaagagtcagggca

SEQ ID NO: 32 - DNA, 100 bp, Cannabis sativa
gaatatcaacgcctagtaggtaagctgacttacctttctcacacgcctaacattagatttgcagtcaagtgttgttggtcaatttatgaatgatcctaaa

SEQ ID NO: 33 - DNA, 100 bp, Cannabis sativa
ctagttggaccatgcatccaggttaacattcacctagcgcatatggttcaattcatcaaattgtcacactagcaatggaaaaatgcttcttcagcatcaa

SEQ ID NO: 34 - DNA, 100 bp, Cannabis sativa
tcaccattacattaggaagtctcacatcaaatcaacatcaaggtgcagcataacattcagcacctgcaccacctaggtgcttaatcttgtttttctccag

SEQ ID NO: 35 - DNA, 100 bp, Cannabis sativa
tgaaacagtacacagatgcaacaattctcaataaaacagtaacaagtggtgaacaaattagttattattgatagtaattgaaaatcagacactagctcta

SEQ ID NO: 36 - DNA, 100 bp, Cannabis sativa
ccaattggatgcaagcacctgaatgaataatatccaaagcttcagaaaacctttgggcagatacatatctgcaataaagcatgcatgcttcatcagtgac

SEQ ID NO: 37 - DNA, 100 bp, Cannabis sativa
tttactagtaagtatgtttttaacatcctaaatatgaatctctaaaacgatgaaacttaaacacatataaagtatgagaaaccttacattagttgcagcg

SEQ ID NO: 38 - DNA, 100 bp, Cannabis sativa
tccaataagcagatctagaatttatcaagtaaattccctaacttattaattcctccttgcaccactatagatttggaattgtactctcgattatatagaa

SEQ ID NO: 39 - DNA, 100 bp, Cannabis sativa
ttccacgatataaatacgctatgagattatccatttgttataatcctaataatcagtgatcctctatagatgatttacaccgagtagggacaaatttatc

SEQ ID NO: 40 - DNA, 100 bp, Cannabis sativa
gattgagatcatttgatctaagatcaactaggtgatattgaattgcatagatattacggtaaatttattatatctattccaagttcaatatcggtccctt

SEQ ID NO: 41 - DNA, 100 bp, Cannabis sativa
gcatgttgatggcatgacaagggaggcgagcttgggggaaattgtgacaaattttcattactttcaaaggagtgcctttttaaggttcaaaataagtact

SEQ ID NO: 42 - DNA, 100 bp, Cannabis sativa
tgcccccccgaaaactgaatggtgtgccatccgtcaacactgctcttgccaccaactggcactaattcatctgagctgcctgcatctgagattgagaggt

SEQ ID NO: 43 - DNA, 100 bp, Cannabis sativa
agcttcagaaacttcaggtttgatacttcgtcaatcttacaagcaatggaaactggtttctctgcatgaattctaacactgctgctctgtaaagttgttt

SEQ ID NO: 44 - DNA, 100 bp, Cannabis sativa
atggagtttatggctgacaaatttgataaaggttgcatcactcgattaaaaatggttgcttcaaccccctttgaacggataacatatacaaaagcagtag

SEQ ID NO: 45 - DNA, 100 bp, Cannabis sativa
atggcggaggtatgagagggattctgtccgggaaagcattggcatacttagagcacgcgctcaaggctaaatcggggaatccagacgctagaatcgctga

SEQ ID NO: 46 - DNA, 100 bp, Cannabis sativa
tgcacaacaagcaggagtttccgttcgtgcgtggggtggaggacctcttggtcctttccattggaacgggtcagctcttggaagtgagtttcgagaacga

SEQ ID NO: 47 - DNA, 100 bp, Cannabis sativa
gtttatggtgcatgagacacagatggccagctgggaatggtcaaagaattttgtcttttactagattttcccatgcatgacaatggtgtaatagctatta

SEQ ID NO: 48 - DNA, 100 bp, Cannabis sativa
ggatcatggcgaccacgggtgattaaatctgtctcaattttatctctagtcactgtatgctgcttcttctggaaaatatttaaggggaaaaaaaagcacc

SEQ ID NO: 49 - DNA, 100 bp, Cannabis sativa
taagctcatacttgaacgtcataaacagctatgagtaagtaaactgcctacagttcccagattagaaaatatgtaaattcaatttgcaaattgataaggg

SEQ ID NO: 50 - DNA, 100 bp, Cannabis sativa
gtgaactagataactaccatcaatcttatttggccatttcctcctatcaatcttatttgatcatttcatctaaaagttctaaattattttgcgataatta

SEQ ID NO: 51 - DNA, 100 bp, Cannabis sativa
gagattctccataagttgtaacacgaaaatgagttccaaaactattcccagctgcctttacttctgtatttcgacagcaaagatattgtaattataattt

SEQ ID NO: 52 - DNA, 100 bp, Cannabis sativa
agtcctatttgtacaattttgaaaaatgcaaggtccattttgttatttaacaaaacatatgatctaattaatattttttacaaaatacaaggtttaaaat

SEQ ID NO: 53 - DNA, 100 bp, Cannabis sativa
catccaacccaagcttactttaaccaatgccctagaaaggacataacacttatccaaggtgcaagtaaacaacattgtaggttatcctatcagttaaacc

SEQ ID NO: 54 - DNA, 100 bp, Cannabis sativa
tgcggtttgtttagcacttctccaaacaactgctcctcccccaagagtaaacaccatcccagatgtagactttctgtcatcaagacaagcctaaaaatct

SEQ ID NO: 55 - DNA, 100 bp, Cannabis sativa
ctaatatagattatttattatctttttaataatttgtttcatattgtattaaaatttataattgtaataattaatataattcaaaattcaatccaaacat

SEQ ID NO: 56 - DNA, 100 bp, Cannabis sativa
ttgtttcttagttaaaaacacagtcataaggtgagaaagcaagaacatttaattaatactagaagtaaaacaagacaatgtgagcttatactagtttata

SEQ ID NO: 57 - DNA, 100 bp, Cannabis sativa
tagtgcagaaagattacctaccataagaattttgttttgacgctgtaattctctacataatgattcaagtttatctcgaacagcaacagctacaacaggc

SEQ ID NO: 58 - DNA, 100 bp, Cannabis sativa
aagaaaagaaaaagtgtagttcagtgttgaggaaaaatctcacaaccaaaattattttgtttcttaatgaccattaactaaacagcctatacttaaggta

SEQ ID NO: 59 - DNA, 100 bp, Cannabis sativa
tcatatcaggtgatgtcatgaatgtcacaactggacctaatgatacagagagacagcatgttgaaagtgataaaagctttactttctttatggtcgaaga

SEQ ID NO: 60 - DNA, 100 bp, Cannabis sativa
agatttttcttacaaaatccaaacccaatctctctcttttctctatacctctctccttgaatgcttctgtggtcgccataaattatgttttcgtggtgga

SEQ ID NO: 61 - DNA, 100 bp, Cannabis sativa
cattgtcttctgattccacactaacttccatctgtgaaccttctccgaaagcttcgttccgtactccatttccacacttacagtgtctttgtttcttatt

SEQ ID NO: 62 - DNA, 100 bp, Cannabis sativa
tcatcatcatcagcatcatctacatcatcatcttcctctggttcatcttctttttcttcttctaacatttgtaataatcaagaattaaaacagttgaaga

SEQ ID NO: 63 - DNA, 100 bp, Cannabis sativa
aaaggaaaacatcagtatattaagaatatgtaaatataaaactgtaatgagatgcttggctataattcaagttttctcatttgcataagcgtgcggatat

SEQ ID NO: 64 - DNA, 100 bp, Cannabis sativa
agtaaaagaatgttagagaaaagcattcagcacgatatcaaataatgaggaggaccatagcagacaataacgaaacctattcttacaagtatagacaaat

SEQ ID NO: 65 - DNA, 100 bp, Cannabis sativa
gccctttgaggataacaattcttagtaatttttttaagtttctctataatctgcataattctatttgccaccggcactgggtgcttccactttgatatac

SEQ ID NO: 66 - DNA, 100 bp, Cannabis sativa
agaggagttatagctccctactattcgagaatgacgagctgtttgtgagctataccttgcctaagatagattatgaggaactagacccaatgactaataa

SEQ ID NO: 67 - DNA, 100 bp, Cannabis sativa
aaaaagagatggactaggagctgggcctggtcaaaatccccaggtctgataataataacatgaagaggttaatggtgccattttattttgcttttagtat

SEQ ID NO: 68 - DNA, 100 bp, Cannabis sativa
tatagtgggctcgccatggaagactgcttagatgtatggctccaaaacctatgaaccgaagtcgttcttttctcatttttgcagtatatatggctagtca

SEQ ID NO: 69 - DNA, 100 bp, Cannabis sativa
atatttatataatgtatgtataaacctataattacaaagatataaatacacagattaagaatcacaccttatgtcacaactatacacattaaataaatct

SEQ ID NO: 70 - DNA, 100 bp, Cannabis sativa
ttgtagcttagcttctgtataccatcatcacatacgatccacttcattaaaaaattattaactataacaaacaaatatttatcagaaaataaaaatcttg

SEQ ID NO: 71 - DNA, 100 bp, Cannabis sativa
agcaagtgaaggaggggtcccacatgtactatataagggattatggggggaacccttttcactttttactttttacttctctcactataaattgaaattt

SEQ ID NO: 72 - DNA, 100 bp, Cannabis sativa
taggatttttgcgccgtttcgatgatctaagcttcccacattcttcctcaacaatttcttgtagtgggtaagctttctattcccgttagttcacttaatt

SEQ ID NO: 73 - DNA, 100 bp, Cannabis sativa
gagaaagcctctcgaagagctattttcagtaattctttggtgattaatagaaagcattcctgtgggaattcattactgtagttgtttgtgttgatatata

SEQ ID NO: 74 - DNA, 100 bp, Cannabis sativa
aagtgttcaatgctggtaagatctttgataatgcttacaaaaatagcactgtcttctataactccaacaaagtaaagttccaaaagtagcttcagagtac

SEQ ID NO: 75 - DNA, 100 bp, Cannabis sativa
gtatctgagtcatcccctgattttccaggaaagaagactttcaaaagcccttgaacaaggctcggggagaagtctttgtatctttgatgaagcaaagagc

SEQ ID NO: 76 - DNA, 100 bp, Cannabis sativa
ttaatagtaaaatgagaagttaaatgtaaaaggatgaactaaatactcgcatatgtcaaaacaaaaacaccaaaaataactaagtataaaattagttcta

SEQ ID NO: 77 - DNA, 100 bp, Cannabis sativa
agggcgatgtcgcgaggtaccgagatctcgcggtgttagtagcagagggtgcctcagtgatgatgggacctccttcctttgtatcatacatttaccttcg

SEQ ID NO: 78 - DNA, 100 bp, Cannabis sativa
atttcttgacctagcttagattttgacatagaaccattcttgaggatactacagtgggttacttagtttgtagagtatgtttatgtgtaccttctaaaga

SEQ ID NO: 79 - DNA, 100 bp, Cannabis sativa
tgagtatatttcctttattttggatttaaaataatacttttttctctagtatctttttgtaatttatacttttaactaatacatttattgtgtgtgtttg

SEQ ID NO: 80 - DNA, 100 bp, Cannabis sativa
catgattctaggagtatggtctttaagtgtttatcgaaaggtgccgttgactttttagtgaaacctattcgaaagaatgagctgaaaaacctttggcaac

SEQ ID NO: 81 - DNA, 100 bp, Cannabis sativa
tgtttgccttctagaattcataaaagacctacagggcggtagtttccaaattctcgacctccttcgagagctcttcttccctcgtctgcctggccttaac

SEQ ID NO: 82 - DNA, 100 bp, Cannabis sativa
acataacaaaaacactatacttccaagctttataatgtgaccatcatggtaaaccagcaaacgcaccatcatgttatttacaaatgaagctaaccatatt

SEQ ID NO: 83 - DNA, 100 bp, Cannabis sativa
tgatgtttcacaagtgaaaggccacaggggttaaataaaaaagaacaaagaaaacaagcattacctgagattctatcatttcctctgagtaatagccatc

SEQ ID NO: 84 - DNA, 100 bp, Cannabis sativa
tccagtgctgggtgacctgggaatgttcgaggcaaatcctgcaaatggaatcacaaactagaagttgggaaaatgcaaagctgctacttataatgagcac

SEQ ID NO: 85 - DNA, 100 bp, Cannabis sativa
agcctctctaagtatggccaactaaaagtcttctcctaagtccacatgaggctcaccaattgttggttcgccaattcctggccctatttccccagcataa

SEQ ID NO: 86 - DNA, 100 bp, Cannabis sativa
ttttacagctaatatacatctaatatctaacactaagaaatatggggaacggagaaatataagttatttcataaggtaaatccattaacaatacattgac

SEQ ID NO: 87 - DNA, 100 bp, Cannabis sativa
gacccaatggtgcataattttgcttggataatacatagatcccatattcacaaacaactagaatgaacaagggaaacacaaacatacaatttgattggat

SEQ ID NO: 88 - DNA, 100 bp, Cannabis sativa
acctttcaatttctttagactttcatttgtttttattctttttgctgagttttagaactcaacatgccactaaactaaattaactaatcttcagtaaaca

SEQ ID NO: 89 - DNA, 100 bp, Cannabis sativa
cctgagaaattaaaccatagtgataatggtgggacagcccctgagttctgtcataagtttagtactcagatcgcattgtcaatatttttcaaagccacta

SEQ ID NO: 90 - DNA, 100 bp, Cannabis sativa
tctatcagaagaagaaaggagagagagaaaaatagatggaatgctgattagggagtttgaaattggagatgttgttagcctttaaataatgggagaagta

SEQ ID NO: 91 - DNA, 100 bp, Cannabis sativa
aagttcaccaacaagttgtttacagatttacaaggtatctgaagatgacacctggtaaagggtcgtacttcaagaagggaactaacaagaacattgagat

SEQ ID NO: 92 - DNA, 100 bp, Cannabis sativa
aataccctaacttcgcataacctgagaggttgcaccaagtgaactgacaccaggtgcccaagtgacaccatgcgccaagtgaattgacaccaggtgccca

SEQ ID NO: 93 - DNA, 100 bp, Cannabis sativa
aaatcatgcacaacttaagcatttgacttagtgatgcaccaatatcacattagcaatccaaaatatgtcaaaatcatgtatttctacgcccaggtgaaca

SEQ ID NO: 94 - DNA, 100 bp, Cannabis sativa
agggaaaattagagtcgaagctctccaagaatgctaagaattactaagttcttgcataaccctcactcagttcacatacattctataagagcaactcaac

SEQ ID NO: 95 - DNA, 100 bp, Cannabis sativa
tgaattggtgataaagtgtaacaagttacatgctaggtgaaaataacatatgacggataccaaatgggcaaagtttgagttgatacaaatgtaggttcgt

SEQ ID NO: 96 - DNA, 100 bp, Cannabis sativa
aattaaatgactccttctactcagatctctaaccattgttcctttctgtcgcagagtatgatcaagatttgagcccgacactccttcagttgtttggatt

SEQ ID NO: 97 - DNA, 100 bp, Cannabis sativa
gctctatatgttccacgatataaatacgctatgagattatccatttgttataatcctaataatcagtgatcctctatagatgatttacaccgagtaggga

SEQ ID NO: 98 - DNA, 100 bp, Cannabis sativa
ttacactcttcaatgtattttatccttaaaacaattagctacatataaatgatatttaagtgatctaatataatcactgaaatgagcactcaatcatata

SEQ ID NO: 99 - DNA, 100 bp, Cannabis sativa
cgatgcatacttatacacccaacccaagtttactttaaccaatgccctggaaatgacataacacttatgcacggtgcaagtaaactacactgtagattat

SEQ ID NO: 100 - DNA, 100 bp, Cannabis sativa
tcatggaaatttgaatttcgaaaagtaactaaatgtgggacttagcgtaattggttgggtgattttactacacgtgtctttatttccttaagattatttt

SEQ ID NO: 101 - DNA, 100 bp, Cannabis sativa
gagaagaatcagagagagcataaaatgacactaaagaatgtagtaatgaggcttttgtcaaacatcagaagatgattcgaaaatggtgaacacaaaaaca

SEQ ID NO: 102 - DNA, 100 bp, Cannabis sativa
acttgttttactactcaccaattagttcttcccgcaatgtttgacggtggcccccctgtatagcggttcaagaattccagtttcgggttttaagtaaatt

SEQ ID NO: 103 - DNA, 100 bp, Cannabis sativa
gcttttagaagaggctgtgaagaatggtaagcagtttgagaacaaggtagagtggggaattgatttggcatctgaacatgaaaggtatgcaaattttatt

SEQ ID NO: 104 - DNA, 100 bp, Cannabis sativa
tacttcgacgtggcagcgggggctggtgttggaggaattttcacggctatgctctttgctacgagtgaccagagccgcccaatatccaaagccgatgata

SEQ ID NO: 105 - DNA, 100 bp, Cannabis sativa
caagtcaaaaagtggaaggccaaggattgggcacgtcccactgcccgtattgctagcgacggctccgctgacctagttgatcaggccgtctccatggcct

SEQ ID NO: 106 - DNA, 100 bp, Cannabis sativa
aatactagatagaattgaaggtgatattgatattgggttggggatgggctaacgtgcgccggagttttgtgtttatgcaattaaatgtcgggtgtagttg

SEQ ID NO: 107 - DNA, 100 bp, Cannabis sativa
aaaaaaacagatttaggcaacacaactcttttagattatcaaccaactccacactcaaactacttcgcgaaaaaagaaatatcaagcagaggattatttt

SEQ ID NO: 108 - DNA, 100 bp, Cannabis sativa
aaatggaaaatggaagaaagaagccttacacgtggacaattcttcacaacacatgtaacaacgccgccaatggaatcccctctcacccggatagtatcta

SEQ ID NO: 109 - DNA, 100 bp, Cannabis sativa
atactaaacttcacgatcagcattcaaaatggtacctgatcaagtgtcaaggcctcatgatcaaccaaccccagcggaagctcaacattgtgtacttgtg

SEQ ID NO: 110 - DNA, 100 bp, Cannabis sativa
atgccaattcagttcaagtcattcagcaaagccaatggtatttactttagatgtaatcatttactttcaagtttgcaaataaagcacacaagaacactta

SEQ ID NO: 111 - DNA, 100 bp, Cannabis sativa
gtatttattgttattgttattgaatattagttgtgctcagtacttttcataggtgattctattagtgatattctttaaaagttatttttttaacattata

SEQ ID NO: 112 - DNA, 100 bp, Cannabis sativa
tgtgtactgataaattataggaatacatttaatcacataatcttaaatactttccactgtgctgacgacacaataaacaagaatatcaatgtgataagaa

SEQ ID NO: 113 - DNA, 100 bp, Cannabis sativa
agtcggtatagcctacgggatttaaagcaccactcttgtagactaacacataatgccttgtacttttcaagtacttcagaatatgcttaactgcagtcca

SEQ ID NO: 114 - DNA, 100 bp, Cannabis sativa
gtattttacttatttaatgcaataggtataccaagcatgccctaagaaaatcaactacaacttatttaaattgttaaaactatattattcaactaacatc

SEQ ID NO: 115 - DNA, 100 bp, Cannabis sativa
actaaagcactctttcaacttttatacaataaaattcattaaaagaagaagtttgcatttcttaagaattacataattgctacttaagaattacatatag

SEQ ID NO: 116 - DNA, 100 bp, Cannabis sativa
gggtgagaccaaaaaatttatcaattagacaaatacgttgctcatattcaaacatactataaaacatggaaaatttaatgtgaaatttattttatgaaaa

SEQ ID NO: 117 - DNA, 100 bp, Cannabis sativa
gcagtcttataaatctcaaaatgccaaaatctctatttatgatgtgatagaataacataaatattcctcattcatcaacatcataaatcacaaatttttg

SEQ ID NO: 118 - DNA, 100 bp, Cannabis sativa
attggtccatagtagacaatcccagagtctttttcattttccccatcttctgctacttttcttgggccaatactgagctttccattgtcttcagctgtag

(19) United States
(12) Patent Application Publication
(10) Pub. No.: US 2024/0049666 A1
(43) Pub. Date: Feb. 15, 2024

(54) MARKER-ASSISTED BREEDING IN CANNABIS PLANTS

(71) Applicant: Central Coast Agriculture, Inc., Buellton, CA (US)

(72) Inventors: DANIEL BARRERA, Buellton, CA (US); ADAM CRISWELL, Buellton, CA (US); JON MYRVOLD, Buellton, CA (US); STEVE BOBZIN, Buellton, CA (US); JOHN DE FRIEL, Buellton, CA (US)

(21) Appl. No.: 18/259,244

(22) PCT Filed: Jan. 28, 2022
(86) PCT No.: PCT/US2022/070402

§ 371 Date: Jun. 23, 2023

Related U.S. Application Data
(60) Provisional application No. 63/142,906, filed on Jan. 28, 2021.

(51) Int. Cl. A01H 1/04; C12Q 1/6895

(52) U.S. Cl. CPC A01H 1/04; C12Q 1/6895; C12Q 2600/156

(57) ABSTRACT

The present invention relates to methods of breeding in Cannabis plants having a Value Phenotype.

### CLAIM OF PRIORITY UNDER 35 U.S.C. § 119

The present application for patent claims priority to Provisional Application No. 63/142,906 entitled “MARKER-ASSISTED BREEDING IN CANNABIS PLANTS” filed Jan. 28, 2021, the entirety of which, including the four Appendices to the Specification as filed, is hereby expressly incorporated by reference herein.

### BACKGROUND

### Field

The present invention relates to methods of marker-assisted breeding in Cannabis plants.

### Background

“Autoflower” or “day-length neutral” Cannabis varieties are those that transition from a vegetative growth stage to a flowering stage based upon age, rather than length-of day. In contrast, most varieties of Cannabis in commercial use transition to the flowering stage based upon the plant's perception of day length, such that the plants flower according to the seasonal variation in day length rather than the age of the plant.

The autoflower trait in Cannabis plants allows for a more consistent crop in terms of growth, yield, and harvest times as compared with day-length sensitive Cannabis varieties. In outdoor Cannabis cultivation, the availability of elite autoflower Cannabis varieties would expand the latitude and planting dates for productive Cannabis cultivation.

### SUMMARY

Embodiments of the invention relate to a method of plant breeding to develop an Autoflower Value Phenotype. The method can include (a) providing a first parent plant, having a phenotype defined as a Value Phenotype, wherein the Value Phenotype comprises at least one trait of interest; (b) providing a second parent plant, having an autoflower phenotype; (c) crossing the first and second parent plants; (d) recovering progeny from the crossing step; (e) screening the progeny for presence of at least one autoflower allele using a marker having at least 51% correlation with presence of the autoflower allele; (f) selecting autoflower carrier progeny, wherein cells of said autoflower carrier progeny comprise at least one autoflower allele; (g) conducting further breeding steps using autoflower carrier progeny crossed with plants having the Value Phenotype; and/or (h) repeating steps e, f, and g until at least one plant having an Autoflower Value Phenotype is obtained.

In some embodiments, the further breeding steps of step f can include at least one of: a backcross; a self-cross; a sibling cross; and creation of a double haploid.

In some embodiments, the method of step e employs one or more markers from Table 1.

Some embodiments of the invention relate to a method of plant breeding to develop a plant with an Autoflower Value Phenotype. The method can include (a) providing a first parent plant, having a phenotype defined as a Value Phenotype, wherein the Value Phenotype comprises at least one trait of interest; (b) providing a second parent plant, having an autoflower phenotype; (c) crossing the first and second parent plants; (d) recovering progeny from the crossing step; (e) identifying one or more loci for which the first and second parent plants are polymorphic such that, for each such polymorphic locus, there exists a first-parent allele and a different second-parent allele; (f) screening individuals of the progeny for presence of (1) at least one autoflower allele (2a) presence of one or more first-parent alleles; and/or (2b) absence one or more second-parent alleles, wherein plants meeting criteria (1) and (2) are designed as desirable progeny; (g) selecting the desirable progeny; (h) conducting further breeding steps using the desirable progeny in one or more of subsequent crosses selected from any of (i) a self-cross of a desirable progeny individual; (ii) a cross between different desirable progeny individuals; (iii) a cross between a desirable progeny individual and the first parent plant; and/or (iv) a cross between a desirable progeny individual and a plant having the Value Phenotype that is not the first parent plant; and/or (i) repeating steps f, g, and h until at least one plant having an Autoflower Value Phenotype is obtained.

In some embodiments, the method of step e employs one or more markers from Table 1.

Some embodiments of the invention relate to a method of plant breeding to develop an Autoflower Value Phenotype. The method can include (a) providing a first parent plant having a phenotype defined as a Value Phenotype, wherein the Value Phenotype comprises at least one trait of interest; (b) providing a second parent plant, having an autoflower phenotype; (c) crossing the first and second parent plants; (d) recovering progeny from the crossing step; (e) screening the progeny phenotypically for presence of at least one autoflower-associated marker and the Value Phenotype; (f) selecting autoflower carrier progeny with the Value Phenotype, wherein cells of said autoflower carrier progeny comprise at least one autoflower-associated marker; (g) conducting further breeding steps using autoflower carrier progeny selfed, sib-mated, or crossed with plants having the Value Phenotype; and/or (h) repeating steps e, f, and g until at least one plant having an Autoflower Value Phenotype is obtained.

Some embodiments of the invention relate to a method for providing a Cannabis plant with a modulated day-length sensitivity phenotype. The method can include (a) selecting an autoflower Cannabis plant, designated as the first Cannabis plant, wherein the selection comprises any of: detecting an autoflower phenotype in a plant, or establishing the presence of an autoflower-associated marker or autoflower-associated genomic sequence; (b) transferring the autoflower-associated marker or autoflower-associated genomic sequence of step a) into a recipient Cannabis plant, thereby conferring a modulated day-length sensitivity phenotype to the recipient Cannabis plant; and/or (c) detecting presence of an autoflower-associated marker in the recipient Cannabis plant. In some embodiments, at least the selecting of step a) and/or the detecting of step c) can include use of a marker indicative of an autoflower allele.

In some embodiments, the transferring of step b can include a cross of the first Cannabis plant with a second Cannabis plant that does not have a modulated day-length sensitivity phenotype, and subsequently selecting a recipient Cannabis plant that has a modulated day-length sensitivity phenotype.

In some embodiments, step a) establishing the presence of the autoflower allele or autoflower-associated genomic sequence in a Cannabis plant can include use of one or more markers from Table 1.

In some embodiments, in any of the methods disclosed herein, the modulated day-length sensitivity phenotype is an autoflower phenotype, attenuation of day-length sensitivity, or increase of day-length sensitivity.

In some embodiments, in any of the methods disclosed herein, the autoflower-associated marker is selected from Table 1.

In some embodiments, in any of the methods disclosed herein, the Value Phenotype can include at least one trait selected from: (a) high THCA accumulation; (b) specific cannabinoid ratio(s); (c) a composition of terpenes and/or other aromatic molecules; (d) monoecy or dioecy (enable or prevent hermaphroditism); (e) branchless or branched architectures with specific height to branch length ratios or total branch length; (f) high flower to leaf ratios that enable pathogen resilience through improved airflow; (g) high flower to leaf ratios that maximize light penetration and flower development in the vertical canopy space; (h) a finished plant height that enables tractor farming inside high tunnels; (i) a finished plant height and flower to leaf ratio that maximizes light penetration all the way to the ground but minimizes total plant height; (j) trichome size; (k) trichome density; (l) advantageous flower structures for oil or flower production; (m) flower diameter length; (n) long or short internodal spacing distance; (o) flower-to-leaf determination ratio (leafiness of flower); (p) metabolites that provide enhanced properties to finished oil products (oxidation resistance, color stability, cannabinoid and terpene stability); (q) specific variants affecting cannabinoid or aromatic molecule biosynthetic pathways; (r) modulators of the flowering time phenotype that increase or decrease maturation time; (s) biomass yield and composition; (t) crude oil yield and composition; (u) resistance to Botrytis, powdery mildew, Fusarium, Pythium, Cladosporium, Alternaria, spider mites, broad mites, russet mites, aphids, nematodes, caterpillars, HLVd or any other Cannabis pathogen or pest of viral, bacterial, fungal, insect, or animal origin; and/or (v) propensity to host specific beneficial and/or endophytic microflora.

Some embodiments of the invention relate to plants, plant parts, tissues, cells, and/or seeds derived from a plant according to any of the methods described herein.

Some embodiments of the invention relate to an allele for providing a modulated day-length sensitivity phenotype to a Cannabis plant, wherein the allele can encode an autoflower protein, wherein the autoflower protein is a protein encoded by a sequence in Table 1.

In some embodiments, the modulation is complete abrogation of day-length sensitivity and the phenotype is autoflower.

In some embodiments, the autoflower phenotype allele can be represented by a coding sequence having at least 35% nucleotide sequence identity with a sequence in Table 1. In some embodiments, the coding sequence can have at least 40, 45, 50, 60, 65, 70, or more percent nucleotide sequence identity with a sequence in Table 1.

Some embodiments of the invention relate to a genomic sequence for providing an autoflower phenotype to a Cannabis plant, wherein the genomic sequence can include 35% nucleotide sequence identity with a sequence in Table 1. In some embodiments, the genomic sequence can have at least 40, 45, 50, 60, 65, 70, or more percent nucleotide sequence identity with a sequence in Table 1.

Some embodiments of the invention relate to a use of a marker for establishing the presence of an autoflower allele or an autoflower-conferring genomic sequence according to any of the methods disclosed herein in a Cannabis plant. In some embodiments, the marker can indicate presence of an allele that encodes an autoflower protein. In some embodiments, the autoflower protein in encoded by a sequence in Table 1.

Some embodiments of the invention relate to a marker indicative of presence of an allele capable of modulating day-length sensitivity in a Cannabis plant. In some embodiments, the marker can be a first marker having a sequence identical to any of the sequences in Table 1 or wherein the marker can be a second marker located in proximity to the first marker, wherein the proximity is sufficient to provide greater than 95% correlation between presence of the second marker and presence of the first marker.

Some embodiments relate to an autoflower Cannabis plant having a Value Phenotype, comprising at least one of the markers described herein.

### BRIEF DESCRIPTION OF THE DRAWINGS

FIG. 1 is a schematic view of the pedigree used in an Example as described.

FIG. 2 is a schematic view of haplotype-blocks

FIG. 3 shows results of a Quantitative Trait Locus (QTL) scan.

FIG. 4 show results from QTL mapping.

### DETAILED DESCRIPTION

Day-length neutral (autoflower) Cannabis varieties typically express less desirable phenotypic characteristics than day-length sensitive Cannabis varieties. For example, lower cannabinoid content, leafy inflorescences and a limited aroma profile are commonly associated with day-length neutral varieties and tend to produce an inferior finished product. There is significant interest in breeding Cannabis to develop autoflower varieties that otherwise have desirable genotypes or phenotypes. Such breeding typically involves a cross of a first, day-length sensitive (photoperiod) parent plant having a desired phenotype (referred to herein as a “Value Phenotype”) with a second parent plant having an autoflower phenotype, whatever other traits it may have. For purposes of this disclosure, a plant expressing all of the desirable features of a given first parent, the Value Phenotype, but in an autoflower form, can be referred to as an “Autoflower Value Phenotype” plant.

The Value Phenotype can include at least one trait selected from one or more of: high THCA accumulation; specific cannabinoid ratio(s); a composition of terpenes and/or other aroma-active and aromatic molecules; monoecy or dioecy (enable or prevent hermaphroditism); branchless or branched architectures with specific height to branch length ratios or total branch length; determinant growth; time to maturity; high flower to leaf ratios that enable pathogen resistance through improved airflow; high flower to leaf ratios that maximize light penetration and flower development in the vertical canopy space; a finished plant height that enables tractor farming inside high tunnels; a finished plant height and flower to leaf ratio that maximizes light penetration all the way to the ground but minimizes total plant height; trichome size; trichome density; advantageous flower structures for oil or flower production (flower diameter length, long or short internodal spacing distance, flower-to-leaf determination ratio (leafiness of flower); metabolites that provide enhanced properties to finished oil products (oxidation resistance, color stability, cannabinoid and terpene stability); specific variants affecting cannabinoid or aromatic molecule biosynthetic pathways; modulators of the flowering time phenotype that increase or decrease maturation time; biomass yield and composition; crude oil yield and composition; resistance to Botrytis, powdery mildew, Fusarium, Pythium, Cladosporium, Alternaria, spider mites, broad mites, russet mites, aphids, nematodes, caterpillars, HLVd or any other Cannabis pathogen or pest of viral, bacterial, fungal, insect, or animal origin; propensity to host specific beneficial and/or endophytic microflora; heavy metal composition in tissues; specific petiole and leaf angles and lengths; and/or the like.

The invention relates to one or more molecular markers and marker-assisted breeding of autoflower Cannabis plants. Detection of a marker and/or other linked marker can be used to identify, select and/or produce plants having the autoflower phenotype and/or to eliminate plants from breeding programs or from planting that do not have the autoflower phenotype. The molecular marker can be utilized to indicate a plant's possession of an autoflower allele well before the trait can be morphologically or functionally manifest in the plant, and also when the plant is heterozygous for the autoflower allele and therefore would never display the autoflower phenotype. Specifically, in the context of breeding to develop Autoflower Value Phenotype varieties, a molecular marker correlating strongly with the autoflower trait can permit very early testing of progeny of a cross to identify those progeny that possess one or more autoflower alleles and discard those individuals that do not. This permits shifting the allele frequency of any plants remaining in the breeding pool, after such screening, to eliminate any plants that do not have at least one autoflower allele. In some embodiments of the invention, the analysis is capable of distinguishing between individuals that are homozygous for the autoflower allele versus those that are heterozygous. In such situations it can be advantageous to discard any heterozygous individuals.

### Definitions

Although the following terms are believed to be well understood by one of ordinary skill in the art, the following definitions are set forth to facilitate understanding of the presently disclosed subject matter.

As used herein, the terms “a” or “an” or “the” can refer to one or more than one. For example, “a” marker (e.g., SNP, QTL, haplotype) can mean one marker or a plurality of markers (e.g., 2, 3, 4, 5, 6, and the like).

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, is meant to encompass, in different embodiments, variations of 20%, 10%, 5%, 1%, 0.5%, or even 0.1% of the specified amount.

As used herein, the transitional phrase “consisting essentially of” means that the scope of a claim is to be interpreted to encompass the specified materials or steps recited in the claim and any others that do not materially affect the basic and novel characteristic(s) of the claimed invention. Thus, the term “consisting essentially of” when used in a claim of this invention is not intended to be interpreted to be equivalent to either “comprising” or “consisting of.”

As used herein, the term “allele” refers to one of two or more different nucleotides or nucleotide sequences that occur at a specific locus.

A “locus” is a position on a chromosome where a gene or marker or allele is located. In some embodiments, a locus can encompass one or more nucleotides.

As used herein, the terms “desired allele,” “target allele” and/or “allele of interest” are used interchangeably to refer to an allele associated with a desired trait. In some embodiments, a desired allele can be associated with either an increase or a decrease (relative to a control) of—or in—a given trait, depending on the nature of the desired phenotype. In some embodiments of this invention, the phrase “desired allele,” “target allele” or “allele of interest” refers to an allele(s) that is associated with autoflower phenotype.

A marker is “associated with” a trait when said trait is linked to it and when the presence of the marker is an indicator of whether and/or to what extent the desired trait or trait form will occur in a plant/germplasm comprising the marker. Similarly, a marker is “associated with” an allele or chromosome interval when it is linked to it and when the presence of the marker is an indicator of whether the allele or chromosome interval is present in a plant/germplasm comprising the marker. For example, “a marker associated with autoflower” refers to a marker whose presence or absence can be used to predict whether a plant will carry an autoflower allele or display an autoflower phenotype.

As used herein, the term “autoflower” or “day length neutral” refers to a plant's ability to transition from a vegetative growth stage to a flowering stage independent of length of day. As used herein, “AF” can be an abbreviation for autoflower.

As used herein, the term “photoperiod sensitivity” refers to the sensitivity of a plant to length of day. Photoperiod sensitive plants will transition from a vegetative growth to a flowering stage based on the plant's perception of length of day. Autoflower plants have low or no photoperiod sensitivity. As used herein, “PP” can be an abbreviation for photoperiod.

As used herein, the terms “backcross” and “backcrossing” refer to the process whereby a progeny plant is crossed back to one of its parents one or more times (e.g., 1, 2, 3, 4, 5, 6, 7, 8, etc.). In a backcrossing scheme, the “donor” parent refers to the parental plant with the desired gene or locus to be introgressed. The “recipient” parent (used one or more times) or “recurrent” parent (used two or more times) refers to the parental plant into which the gene or locus is being introgressed. For example, see Ragot, M. et al. Marker-assisted Backcrossing: A Practical Example, in TECHNIQUES ET UTILISATIONS DES MARQUEURS MOLECULAIRES LES COLLOQUES, Vol. 72, pp. 45-56 (1995); and Openshaw et al., Marker-assisted Selection in Backcross Breeding, in PROCEEDINGS OF THE SYMPOSIUM “ANALYSIS OF MOLECULAR MARKER DATA” pp. 41-43 (1994). The initial cross gives rise to the F1 generation. The term “BC1” refers to the second use of the recurrent parent, “BC2” refers to the third use of the recurrent parent, and so on.

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

As used herein, the terms “cultivar” and “variety” refer to a group of similar plants that by structural or genetic features and/or performance can be distinguished from other varieties within the same species.

As used herein, the terms “elite” and/or “elite line” refer to any line that is substantially homozygous and has resulted from breeding and selection for desirable agronomic performance.

As used herein, the terms “exotic,” “exotic line” and “exotic germplasm” refer to any plant, line or germplasm that is not elite. In general, exotic plants/germplasms are not derived from any known elite plant or germplasm, but rather are selected to introduce one or more desired genetic elements into a breeding program (e.g., to introduce novel alleles into a breeding program).

A “genetic map” is a description of genetic linkage relationships among loci on one or more chromosomes within a given species, generally depicted in a diagrammatic or tabular form. For each genetic map, distances between loci are measured by the recombination frequencies between them. Recombination between loci can be detected using a variety of markers. A genetic map is a product of the mapping population, types of markers used, and the polymorphic potential of each marker between different populations. The order and genetic distances between loci can differ from one genetic map to another.

As used herein, the term “genotype” refers to the genetic constitution of an individual (or group of individuals) at one or more genetic loci, as contrasted with the observable and/or detectable and/or manifested trait (the phenotype). Genotype is defined by the allele(s) of one or more known loci that the individual has inherited from its parents. The term genotype can be used to refer to an individual's genetic constitution at a single locus, at multiple loci, or more generally, the term genotype can be used to refer to an individual's genetic make-up for all the genes in its genome. Genotypes can be indirectly characterized. e.g., using markers and/or directly characterized by nucleic acid sequencing.

As used herein, the term “germplasm” refers to genetic material of or from an individual (e.g., a plant), a group of individuals (e.g., a plant line, variety or family), or a clone derived from a line, variety, species, or culture. The germplasm can be part of an organism or cell, or can be separate from the organism or cell. In general, germplasm provides genetic material with a specific genetic makeup that provides a foundation for some or all of the hereditary qualities of an organism or cell culture. As used herein, germplasm includes cells, seed or tissues from which new plants can be grown, as well as plant parts that can be cultured into a whole plant (e.g., leaves, stems, buds, roots, pollen, cells, etc.).

A “haplotype” is the genotype of an individual at a plurality of genetic loci, i.e., a combination of alleles. Typically, the genetic loci that define a haplotype are physically and genetically linked, i.e., on the same chromosome segment. The term “haplotype” can refer to polymorphisms at a particular locus, such as a single marker locus, or polymorphisms at multiple loci along a chromosomal segment.

As used herein, the term “heterozygous” refers to a genetic status wherein different alleles reside at corresponding loci on homologous chromosomes.

As used herein, the term “homozygous” refers to a genetic status wherein identical alleles reside at corresponding loci on homologous chromosomes.

As used herein, the term “hybrid” in the context of plant breeding refers to a plant that is the offspring of genetically dissimilar parents produced by crossing plants of different lines or breeds or species, including but not limited to the cross between two inbred lines.

As used herein, the term “inbred” refers to a substantially homozygous plant or variety. The term can refer to a plant or plant variety that is substantially homozygous throughout the entire genome or that is substantially homozygous with respect to a portion of the genome that is of particular interest.

As used herein, the term “indel” refers to an insertion or deletion in a pair of nucleotide sequences, wherein a first sequence can be referred to as having an insertion relative to a second sequence or the second sequence can be referred to as having a deletion relative to the first sequence.

As used herein, the terms “introgression,” “introgressing” and “introgressed” refer to both the natural and artificial transmission of a desired allele or combination of desired alleles of a genetic locus or genetic loci from one genetic background to another. For example, a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be a selected allele of a marker, a QTL, a transgene, or the like. Offspring comprising the desired allele can be backcrossed one or more times (e.g., 1, 2, 3, 4, or more times) to a line having a desired genetic background, selecting for the desired allele, with the result being that the desired allele becomes fixed in the desired genetic background. For example, a marker associated with metribuzin tolerance can be introgressed from a donor into a recurrent parent that is metribuzin intolerant. The resulting offspring could then be backcrossed one or more times and selected until the progeny possess the genetic marker(s) associated with metribuzin tolerance in the recurrent parent background.

As used herein, the term “linkage” refers to the degree with which one marker locus is associated with another marker locus or some other. The linkage relationship between a genetic marker and a phenotype can be given as a “probability” or “adjusted probability.” Linkage can be expressed as a desired limit or range. For example, in some embodiments, any marker is linked (genetically and physically) to any other marker when the markers are separated by less than about 50, 40, 30, 25, 20, or 15 map units (or cM).

A centimorgan (“cM”) or a genetic map unit (m.u.) is a unit of measure of recombination frequency and is defined as the distance between genes for which 1 product of meiosis in 100 is recombinant. One cM is equal to a 1% chance that a marker at one genetic locus will be separated from a marker at a second locus due to crossing over in a single generation. Thus, a recombinant frequency (RF) of 1% is equivalent to 1 m.u. or cM.

As used herein, the phrase “linkage group” refers to all of the genes or genetic traits that are located on the same chromosome. Within the linkage group, those loci that are close enough together can exhibit linkage in genetic crosses. Since the probability of crossover increases with the physical distance between loci on a chromosome, loci for which the locations are far removed from each other within a linkage group might not exhibit any detectable linkage in direct genetic tests. The term “linkage group” is mostly used to refer to genetic loci that exhibit linked behavior in genetic systems where chromosomal assignments have not yet been made. Thus, the term “linkage group” is, in common usage and in many embodiments, synonymous with the physical entity of a chromosome, although one of ordinary skill in the art will understand that a linkage group can also be defined as corresponding to a region of (i.e., less than the entirety) of a given chromosome.

As used herein, the term “linkage disequilibrium” refers to a non-random segregation of genetic loci or traits (or both). In either case, linkage disequilibrium implies that the relevant loci are within sufficient physical proximity along a length of a chromosome so that they segregate together with greater than random (i.e., non-random) frequency (in the case of co-segregating traits, the loci that underlie the traits are in sufficient proximity to each other). Markers that show linkage disequilibrium are considered linked. Linked loci co-segregate more than 50% of the time, e.g., from about 51% to about 100% of the time. In other words, two markers that co-segregate have a recombination frequency of less than 50% (and, by definition, are separated by less than 50 cM on the same chromosome). As used herein, linkage can be between two markers, or alternatively between a marker and a phenotype. A marker locus can be “associated with” (linked to) a trait, e.g., metribuzin tolerance. The degree of linkage of a genetic marker to a phenotypic trait is measured, e.g., as a statistical probability of co-segregation of that marker with the phenotype.

Linkage disequilibrium is most commonly assessed using the measure r², which is calculated using the formula described by Hill and Robertson, Theor. Appl. Genet. 38:226 (1968). When r²⁼¹, complete linkage disequilibrium exists between the two marker loci, meaning that the markers have not been separated by recombination and have the same allele frequency. Values for r² above ⅓ indicate sufficiently strong linkage disequilibrium to be useful for mapping. Ardlie et al., Nature Reviews Genetics 3:299 (2002). Hence, alleles are in linkage disequilibrium when r² values between pairwise marker loci are greater than or equal to about 0.3, 0.4, 0.5, 0.6, 0.7, 0.8, 0.9, or 1.0.

As used herein, the term “linkage equilibrium” describes a situation where two markers independently segregate, i.e., sort among progeny randomly. Markers that show linkage equilibrium are considered unlinked (whether or not they lie on the same chromosome).

As used herein, the terms “marker” and “genetic marker” are used interchangeably to refer to a nucleotide and/or a nucleotide sequence. A marker can be, but is not limited to, an allele, a gene, a haplotype, a chromosome interval, a restriction fragment length polymorphism (RFLP), a simple sequence repeat (SSR), a random amplified polymorphic DNA (RAPD), a cleaved amplified polymorphic sequence (CAPS) (Rafalski and Tingey, Trends in Genetics 9:275 (1993)), an amplified fragment length polymorphism (AFLP) (Vos et al., Nucleic Acids Res. 23:4407 (1995)), a single nucleotide polymorphism (SNP) (Brookes, Gene 234:177 (1993)), a sequence-characterized amplified region (SCAR) (Paran and Michelmore, Theor. Appl. Genet. 85:985 (1993)), a sequence-tagged site (STS) (Onozaki et al., Euphytica 138:255 (2004)), a single-stranded conformation polymorphism (SSCP) (Orita et al., Proc. Natl. Acad. Sci. USA 86:2766 (1989)), an inter-simple sequence repeat (ISSR) (Blair et al., Theor. Appl. Genet. 98:780 (1999)), an inter-retrotransposon amplified polymorphism (IRAP), a retrotransposon-microsatellite amplified polymorphism (REMAP) (Kalendar et al., Theor. Appl. Genet. 98:704 (1999)), an isozyme marker, an RNA cleavage product (such as a Lynx tag) or any combination of the markers described herein. A marker can be present in genomic or expressed nucleic acids (e.g., ESTs). A number of Cannabis genetic markers are known in the art, and are published or available from various sources. In some embodiments, a genetic marker of this invention is an SNP allele, a SNP allele located in a chromosome interval and/or a haplotype (combination of SNP alleles) each of which is associated with an autoflower phenotype.

As used herein, the term “background marker” refers to markers throughout a genome that are polymorphic between a recurrent parent and a donor parent, and that are not known to be associated with a trait sought to be introgressed from a donor parent genome to the recurrent parent genome.

Markers corresponding to genetic polymorphisms between members of a population can be detected by methods well-established in the art. These include, but are not limited to, nucleic acid sequencing, hybridization methods, amplification methods (e.g., PCR-based sequence specific amplification methods), detection of restriction fragment length polymorphisms (RFLP), detection of isozyme markers, detection of polynucleotide polymorphisms by allele specific hybridization (ASH), detection of amplified variable sequences of the plant genome, detection of self-sustained sequence replication, detection of simple sequence repeats (SSRs), detection of randomly amplified polymorphic DNA (RAPD), detection of single nucleotide polymorphisms (SNPs), and/or detection of amplified fragment length polymorphisms (AFLPs). Thus, in some embodiments of this invention, such well known methods can be used to detect the SNP alleles as defined herein.

Accordingly, in some embodiments of this invention, a marker is detected by amplifying a Glycine sp. nucleic acid with two oligonucleotide primers by, for example, the polymerase chain reaction (PCR).

A “marker allele,” also described as an “allele of a marker locus,” can refer to one of a plurality of polymorphic nucleotide sequences found at a marker locus in a population that is polymorphic for the marker locus.

“Marker-assisted selection” (MAS) or “marker-assisted breeding” is a process by which phenotypes are selected based on marker genotypes. Marker assisted selection/breeding includes the use of marker genotypes for identifying plants for inclusion in and/or removal from a breeding program or planting.

As used herein, the terms “marker locus” and “marker loci” refer to a specific chromosome location or locations in the genome of an organism where a specific marker or markers can be found. A marker locus can be used to track the presence of a second linked locus, e.g., a linked locus that encodes or contributes to expression of a phenotypic trait. For example, a marker locus can be used to monitor segregation of alleles at a locus, such as a QTL or single gene, that are genetically or physically linked to the marker locus.

As used herein, the terms “marker probe” and “probe” refer to a nucleotide sequence or nucleic acid molecule that can be used to detect the presence of one or more particular alleles within a marker locus (e.g., a nucleic acid probe that is complementary to all of or a portion of the marker or marker locus, through nucleic acid hybridization). Marker probes comprising about 8, 10, 15, 20, 30, 40, 50, 60, 70, 80, 90, 100 or more contiguous nucleotides can be used for nucleic acid hybridization. Alternatively, in some aspects, a marker probe refers to a probe of any type that is able to distinguish (i.e., genotype) the particular allele that is present at a marker locus.

As used herein, the term “molecular marker” can be used to refer to a genetic marker, as defined above, or an encoded product thereof (e.g., a protein) used as a point of reference when identifying a linked locus. A molecular marker can be derived from genomic nucleotide sequences or from expressed nucleotide sequences (e.g., from a spliced RNA, a cDNA, etc.). The term also refers to nucleotide sequences complementary to or flanking the marker sequences, such as nucleotide sequences used as probes and/or primers capable of amplifying the marker sequence. Nucleotide sequences are “complementary” when they specifically hybridize in solution, e.g., according to Watson-Crick base pairing rules. Some of the markers described herein can also be referred to as hybridization markers when located on an indel region. This is because the insertion region is, by definition, a polymorphism vis-ã-vis a plant without the insertion. Thus, the marker need only indicate whether the indel region is present or absent. Any suitable marker detection technology can be used to identify such a hybridization marker, e.g., SNP technology.

As used herein, the term “primer” refers to an oligonucleotide which is capable of annealing to a nucleic acid target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some embodiments an extension primer and in some embodiments an amplification primer) is in some embodiments single stranded for maximum efficiency in extension and/or amplification. In some embodiments, the primer is an oligodeoxyribonucleotide. A primer is typically sufficiently long to prime the synthesis of extension and/or amplification products in the presence of the agent for polymerization. The minimum lengths of the primers can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification. As such, it will be understood that the term “primer”, as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing.

Primers can be prepared by any suitable method. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066.

Primers can be labeled, if desired, by incorporating detectable moieties by for instance spectroscopic, fluorescence, photochemical, biochemical, immunochemical, or chemical moieties.

The PCR method is well described in handbooks and known to the skilled person. After amplification by PCR, target polynucleotides can be detected by hybridization with a probe polynucleotide which forms a stable hybrid with that of the target sequence under stringent to moderately stringent hybridization and wash conditions. If it is expected that the probes are essentially completely complementary (i.e., about 99% or greater) to the target sequence, stringent conditions can be used. If some mismatching is expected, for example if variant strains are expected with the result that the probe will not be completely complementary, the stringency of hybridization can be reduced. In some embodiments, conditions are chosen to rule out non-specific/adventitious binding. Conditions that affect hybridization, and that select against non-specific binding are known in the art, and are described in, for example, Sambrook & Russell (2001). Molecular Cloning: A Laboratory Manual, Third Edition, Cold Spring Harbor Laboratory Press, Cold Spring Harbor, N.Y., United States of America. Generally, lower salt concentration and higher temperature hybridization and/or washes increase the stringency of hybridization conditions.

As used herein, the term “probe” refers to a single-stranded oligonucleotide sequence that will form a hydrogen-bonded duplex with a complementary sequence in a target nucleic acid sequence analyte or its cDNA derivative.

Different nucleotide sequences or polypeptide sequences having homology are referred to herein as “homologues.” The term homologue includes homologous sequences from the same and other species and orthologous sequences from the same and other species. “Homology” refers to the level of similarity between two or more nucleotide sequences and/or amino acid sequences in terms of percent of positional identity (i.e., sequence similarity or identity). Homology also refers to the concept of similar functional properties among different nucleic acids, amino acids, and/or proteins.

As used herein, the phrase “nucleotide sequence homology” refers to the presence of homology between two polynucleotides. Polynucleotides have “homologous” sequences if the sequence of nucleotides in the two sequences is the same when aligned for maximum correspondence. The “percentage of sequence homology” for polynucleotides, such as 50, 55, 60, 65, 70, 75, 80, 85, 90, 95, 96, 97, 98, 99 or 100 percent sequence homology, can be determined by comparing two optimally aligned sequences over a comparison window (e.g., about 20-200 contiguous nucleotides), wherein the portion of the polynucleotide sequence in the comparison window can include additions or deletions (i.e., gaps) as compared to a reference sequence for optimal alignment of the two sequences. Optimal alignment of sequences for comparison can be conducted by computerized implementations of known algorithms, or by visual inspection. Readily available sequence comparison and multiple sequence alignment algorithms are, respectively, the Basic Local Alignment Search Tool (BLAST®; Altschul et al. (1990) J Mol Biol 215:403-10; Altschul et al. (1997) Nucleic Acids Res 25:3389-3402) and ClustalX (Chenna et al. (2003) Nucleic Acids Res 31:3497-3500) programs, both available on the Internet. Other suitable programs include, but are not limited to, GAP, BestFit, PlotSimilarity, and FASTA, which are part of the Accelrys GCG Package available from Accelrys Software, Inc. of San Diego, Calif., United States of America.

As used herein “sequence identity” refers to the extent to which two optimally aligned polynucleotide or polypeptide sequences are invariant throughout a window of alignment of components, e.g., nucleotides or amino acids. “Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “substantially identical” or “corresponding to” means that two nucleotide sequences have at least 50%, 60%, 70%, 75%, 80%, 85%, 90% or 95% sequence identity. In some embodiments, the two nucleotide sequences can have at least 85%, 90%, 95%, 96%, 97%, 98%, 99% or 100% sequence identity.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned (with appropriate nucleotide insertions, deletions, or gaps totaling less than 20 percent of the reference sequence over the window of comparison). In some embodiments, “percent identity” can refer to the percentage of identical amino acids in an amino acid sequence.

Optimal alignment of sequences for aligning a comparison window is well known to those skilled in the art and can be conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., Burlington, Mass.). The comparison of one or more polynucleotide sequences can be to a full-length polynucleotide sequence or a portion thereof, or to a longer polynucleotide sequence. For purposes of this invention “percent identity” can also be determined using BLAST® X version 2.0 for translated nucleotide sequences and BLAST® N version 2.0 for polynucleotide sequences.

The percent of sequence identity can be determined using the “Best Fit” or “Gap” program of the Sequence Analysis Software Package™ (Version 10; Genetics Computer Group, Inc., Madison, Wis.). “Gap” utilizes the algorithm of Needleman and Wunsch (Needleman and Wunsch, J Mol. Biol. 48:443-453, 1970) to find the alignment of two sequences that maximizes the number of matches and minimizes the number of gaps. “BestFit” performs an optimal alignment of the best segment of similarity between two sequences and inserts gaps to maximize the number of matches using the local homology algorithm of Smith and Waterman (Smith and Waterman, Adv. Appl. Math., 2:482-489, 1981, Smith et al., Nucleic Acids Res. 11:2205-2220, 1983).

Useful methods for determining sequence identity are also disclosed in Guide to Huge Computers (Martin J. Bishop, ed., Academic Press, San Diego (1994)), and Carillo et al. (Applied Math 48:1073 (1988)). More particularly, preferred computer programs for determining sequence identity include but are not limited to the Basic Local Alignment Search Tool (BLAST®) programs which are publicly available from National Center Biotechnology Information (NCBI) at the National Library of Medicine, National Institute of Health, Bethesda, Md. 20894; see BLAST® Manual, Altschul et al., NCBI, NLM, NIH; (Altschul et al., J. Mol. Biol. 215:403-410 (1990)); version 2.0 or higher of BLAST® programs allows the introduction of gaps (deletions and insertions) into alignments; for peptide sequence BLAST® X can be used to determine sequence identity; and for polynucleotide sequence BLAST® N can be used to determine sequence identity.

As used herein, the terms “phenotype,” “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait.” In other cases, a phenotype is the result of several genes.

As used herein, the term “polymorphism” refers to a variation in the nucleotide sequence at a locus, where said variation is too common to be due merely to a spontaneous mutation. A polymorphism must have a frequency of at least about 1% in a population. A polymorphism can be a single nucleotide polymorphism (SNP), or an insertion/deletion polymorphism, also referred to herein as an “indel.” Additionally, the variation can be in a transcriptional profile or a methylation pattern. The polymorphic site or sites of a nucleotide sequence can be determined by comparing the nucleotide sequences at one or more loci in two or more germplasm entries.

As used herein, the term “plant” can refer to a whole plant, any part thereof, or a cell or tissue culture derived from a plant. Thus, the term “plant” can refer, as indicated by context, to a whole plant, a plant component or a plant organ (e.g., leaves, stems, roots, etc.), a plant tissue, a seed and/or a plant cell. A plant cell is a cell of a plant, taken from a plant, or derived through culture from a cell taken from a plant.

The term “Cannabis” or “cannabis” refers to a genus of flowering plants in the family Cannabaceae. Cannabis is an annual, dioecious, flowering herb that, by some taxonomic approaches, includes, but is not limited to three different species. Cannabis sativa, Cannabis indica and Cannabis ruderalis. Other taxonomists argue that the genus Cannabis is monospecific, and use sativa as the species name. The genus Cannabis is inclusive.

As used herein, the term “plant part” includes but is not limited to embryos, pollen, seeds, leaves, flowers (including but not limited to anthers, ovules and the like), fruit, stems or branches, roots, root tips, cells including cells that are intact in plants and/or parts of plants, protoplasts, plant cell tissue cultures, plant calli, plant clumps, and the like. Thus, a plant part includes Cannabis tissue culture from which Cannabis plants can be regenerated. Further, as used herein, “plant cell” refers to a structural and physiological unit of the plant, which comprises a cell wall and also can refer to a protoplast. A plant cell of the present invention can be in the form of an isolated single cell or can be a cultured cell or can be a part of a higher-organized unit such as, for example, a plant tissue or a plant organ.

As used herein, the term “population” refers to a genetically heterogeneous collection of plants sharing a common genetic derivation.

As used herein, the terms “progeny”, “progeny plant,” and/or “offspring” refer to a plant generated from a vegetative or sexual reproduction from one or more parent plants. A progeny plant can be obtained by cloning or selfing a single parent plant, or by crossing two parental plants and includes selfings as well as the F1 or F2 or still further generations. An F1 is a first-generation offspring produced from parents at least one of which is used for the first time as donor of a trait, while offspring of second generation (F2) or subsequent generations (F3, F4, and the like) are specimens produced from selfings or crossings of F1s, F2s and the like. An F1 can thus be (and in some embodiments is) a hybrid resulting from a cross between two true breeding parents (the phrase “true-breeding” refers to an individual that is homozygous for one or more traits), while an F2 can be (and in some embodiments is) an offspring resulting from self-pollination of the F1 hybrids.

As used herein, the term “reference sequence” refers to a defined nucleotide sequence used as a basis for nucleotide sequence comparison. The reference sequence for a marker, for example, can be obtained by genotyping a number of lines at the locus or loci of interest, aligning the nucleotide sequences in a sequence alignment program, and then obtaining the consensus sequence of the alignment. Hence, a reference sequence identifies the polymorphisms in alleles at a locus. A reference sequence need not be a copy of an actual nucleic acid sequence from a relevant organism; however, a reference sequence is useful for designing primers and probes for actual polymorphisms in the locus or loci.

### Genetic Mapping

Genetic loci correlating with particular phenotypes, such as photoperiod sensitivity, can be mapped in an organism's genome. By identifying a marker or cluster of markers that co-segregate with a trait of interest, the breeder is able to rapidly select a desired phenotype by selecting for the proper marker (a process called marker-assisted selection). Such markers can also be used by breeders to design genotypes in silico and to practice whole genome selection.

The present invention provides markers associated with autoflower. Detection of these markers and/or other linked markers can be used to identify, select and/or produce plants having the autoflower phenotype and/or to eliminate plants from breeding programs or from planting that do not have the autoflower phenotype.

Markers Associated with Autoflower

Molecular markers are used for the visualization of differences in nucleic acid sequences. This visualization can be due to DNA-DNA hybridization techniques after digestion with a restriction enzyme (e.g., an RFLP) and/or due to techniques using the polymerase chain reaction (e.g., SNP, STS, SSR/microsatellites, AFLP, and the like). In some embodiments, all differences between two parental genotypes segregate in a mapping population based on the cross of these parental genotypes. The segregation of the different markers can be compared and recombination frequencies can be calculated. Methods for mapping markers in plants are disclosed in, for example, Glick & Thompson (1993) Methods in Plant Molecular Biology and Biotechnology, CRC Press, Boca Raton, Florida, United States of America; Zietkiewicz et al. (1994) Genomics 20:176-183.

The recombination frequencies of genetic markers on different chromosomes and/or in different linkage groups are generally 50%. Between genetic markers located on the same chromosome or in the same linkage group, the recombination frequency generally depends on the physical distance between the markers on a chromosome. A low recombination frequency typically corresponds to a low genetic distance between markers on a chromosome. Comparison of all recombination frequencies among a set of genetic markers results in the most logical order of the genetic markers on the chromosomes or in the linkage groups. This most logical order can be depicted in a linkage map. A group of adjacent or contiguous markers on the linkage map that is associated with a trait of interest can provide the position of a locus associated with that trait.

Table 1 provides information about autoflower associated markers. Markers of the present invention are described herein with respect to the positions of marker loci in the Cannabis sativa cs10 GenBank assembly accession: GCA_900626175.2 (Assembly [Internet]. Bethesda (MD): National Library of Medicine (US), National Center for Biotechnology Information; 2012-2022 Jan. 24. Accession No. GCA_900626175.2, cs10; Available from: www<dot>ncbi<dot>nlm<dot>nih<dot>gov/assembly/GCA_900626175.2).

TABLE 1

| Marker_ | | | | | Marker_ | | SEQ | Left_ |
|---|---|---|---|---|---|---|---|---|
| Num | Chrom | Pos | Ref | Alt | Allele | Left_Seq | ID NO | GC |
| M01 | 1 | 19351704 | A | G | A | acaagaacaagtataatatagtcgaga | 1 | 33 |
| | | | | | | atgattctctgttgagttctctcaaagtg | | |
| | | | | | | attcaactctcacattcttacccaaaaat | | |
| | | | | | | cttctttttctacag | | |
| M02 | 1 | 19353247 | A | G | A | caactgataaccttctaaatctgtctgta | 2 | 37 |
| | | | | | | tgaatccttttgacacctttatttggtcttc | | |
| | | | | | | gttatcttgttctttcggctccacaacaa | | |
| | | | | | | cttttgtcta | | |
| M03 | 1 | 19402679 | T | A | A | gtaacactgatcaagtagatggtggtg | 3 | 38 |
| | | | | | | gtcgccatagaagatcattctctttggc | | |
| | | | | | | ttttttaagatattcaacatacaagtcca | | |
| | | | | | | gttcatcttcttcttc(etc) | | |
| M04 | 1 | 19412546 | G | A | G | gggagttcttcagcaatgtcaagagct | 4 | 42 |
| | | | | | | gttttatggtctctagtcaatgcattaac | | |
| | | | | | | attggtgtctggaaggagtaacaactc | | |
| | | | | | | ctttactatctgcaagg | | |
| M05 | 1 | 19413329 | A | G | A | gatctaatagcacttggacaattgctgc | 5 | 35 |
| | | | | | | aggaaagtaagtgccaacatgagttta | | |
| | | | | | | gaagttaatgtagaagtccattttatttg | | |
| | | | | | | attaaagacacttcct | | |
| M06 | 1 | 19682959 | G | T | T | cagccaattgaaactttctgcaagtaca | 6 | 37 |
| | | | | | | tgttctgtatacaatatccaccacacag | | |
| | | | | | | atcacattattccctggttaatgcactta | | |
| | | | | | | aaacttgtttgcatc | | |
| M07 | 1 | 19687541 | C | T | C | taaataccccatgaggggcctgaatg | 7 | 57 |
| | | | | | | gttggggcttgcatcaccggagcggtt | | |
| | | | | | | agagccagaggtggtattttgggagct | | |
| | | | | | | tgaagaaggcccaccccctg | | |
| M08 | 1 | 19692966 | G | C | C | tgtgttaaatgtagaatgttttctaagag | 8 | 45 |
| | | | | | | gaatggttatgttggggaacaaccttg | | |
| | | | | | | acagggagacaccaagggatctggt | | |
| | | | | | | aggggtggtgatggttctg | | |
| M09 | 1 | 19696755 | G | T | T | caaaggctctctttcccattgggtagg | 9 | 38 |
| | | | | | | gatttatttttacctggttaatgatcttact | | |
| | | | | | | catctgcagttgtgtaatgtaaatcactt | | |
| | | | | | | gtctctcttcgta | | |
| M10 | 1 | 19713019 | T | C | C | ccattgcccaacctttataaatctttcaa | 10 | 37 |
| | | | | | | ttgtacctattccacagtcaggcaaact | | |
| | | | | | | atgtctataatatcagtttacatggatcc | | |
| | | | | | | acccacttactttc | | |
| M11 | 1 | 19713824 | G | A | A | gatggtagtgatacatcgggtacatcg | 11 | 54 |
| | | | | | | gcatcaatgtgttggcgctcatcacac | | |
| | | | | | | cagggtatacttgagagtagtgccctc | | |
| | | | | | | ctcccatgtaggccccacc | | |
| M12 | 1 | 19717871 | G | A | A | gttgttgcattttaagccttttgaaatattt | 12 | 29 |
| | | | | | | gtctagagtctctgatctcattttctatag | | |
| | | | | | | taaactaactgctttatttgtttctttgttgt | | |
| | | | | | | gtatgta | | |
| M13 | 1 | 19718025 | T | C | C | ccttttcactttttactttttacttctctcact | 13 | 36 |
| | | | | | | ataaattgaaatttgaagaagctcttcct | | |
| | | | | | | tctccatagaggaccaaacccacaca | | |
| | | | | | | agcatcattctc | | |
| M14 | 1 | 19807569 | G | A | A | atgcatagggaatcagaactcagtttta | 14 | 37 |
| | | | | | | gttatgttgaaagggtttagaattgaga | | |
| | | | | | | aatctcttgggagagatctagccttctt | | |
| | | | | | | gaaaggttagaatagg | | |
| M15 | 1 | 19812569 | T | C | C | acacctcttgcccagagagtggaagc | 15 | 45 |
| | | | | | | ccaagaaatattcttccctgtcgagcaa | | |
| | | | | | | aactagcaaggagagataaatttgtct | | |
| | | | | | | gagtagtatcccgatcctt | | |
| M16 | 1 | 19812701 | T | C | C | tgcttacaaaaatagcactgtcttctata | 16 | 37 |
| | | | | | | actccaacaaagtaaagttccaaaagt | | |
| | | | | | | agcttcagagtactgcgcttcttcatag | | |
| | | | | | | ccttttgattcctatc | | |
| M17 | 1 | 19950755 | T | C | T | tctcaacaaacaggttcggaacagtaa | 17 | 27 |
| | | | | | | aaaattgatggattcattctatttataaa | | |
| | | | | | | gcaaaaataataacaagtgttataacg | | |
| | | | | | | agatttctaagatcaat | | |
| M18 | 1 | 19958734 | C | G | G | tttctagtctttggttcttccaggtggag | 18 | 38 |
| | | | | | | attatttactttgtcttcatagtggaaaatt | | |
| | | | | | | tgggattttggactcacagagttagctc | | |
| | | | | | | acttctcctttc | | |
| M19 | 1 | 19988215 | G | A | G | tactctggtgcttcacgtgtctatttgtg | 19 | 37 |
| | | | | | | ctattttgatgttcatatttatagtctagc | | |
| | | | | | | gggaagttttttagtcatttcgttcatga | | |
| | | | | | | agggtcaagtac | | |
| M20 | 1 | 19988827 | G | T | T | gatctcgttttgactgaggtagtcatgc | 20 | 41 |
| | | | | | | cctgtttatctggtattggtcttctaggc | | |
| | | | | | | aagatcatgagcaaaaaaacatgcaa | | |
| | | | | | | ggacatccctgtaatta | | |
| M21 | 1 | 19988955 | A | T | A | aaaataatacttttttctctagtatctttttg | 21 | 24 |
| | | | | | | taatttatacttttaactaatacatttattgt | | |
| | | | | | | gtgtgtttgtgtttgtgttttcacagtgat | | |
| | | | | | | gtcttc | | |
| M22 | 1 | 19994256 | G | T | G | tgaggaattggccaccccaaggcttttt | 22 | 48 |
| | | | | | | ctagttgcctagcccgcgcagtaatta | | |
| | | | | | | agataagccttcttggagtctccgagg | | |
| | | | | | | taatcaaaattgcctgca | | |
| M23 | 1 | 20011591 | A | T | A | tctgctctgtgatgggatcctgtacctc | 23 | 41 |
| | | | | | | ctcattagtacactttccatcattcgttgt | | |
| | | | | | | catctcctcagatttcacctgtttcattca | | |
| | | | | | | agcaaatattag | | |
| M24 | 1 | 20015613 | G | A | G | cacaaacccagtttaggaaacctctca | 24 | 39 |
| | | | | | | cgcaccaactcctcaaaaacgagttg | | |
| | | | | | | atctacctgacaaaatacaacaatattc | | |
| | | | | | | aacagaactctcatttttc | | |
| M25 | 1 | 20016226 | G | A | G | ttaaccaaggtatccaaaccaatacct | 25 | 44 |
| | | | | | | ggcaatatccaacagagggattatgtc | | |
| | | | | | | ttgcataggcagtaagtagacgcctca | | |
| | | | | | | aggcatttctaccatcctc | | |
| M26 | 1 | 20030132 | C | T | C | atgtagtcctggtcatccatccatctca | 26 | 45 |
| | | | | | | ggaatttgcgtctctgttgggtgaatgc | | |
| | | | | | | atcacaacctacaacacttggttcttca | | |
| | | | | | | gtttggacttggctta | | |
| M27 | 1 | 20712699 | A | C | A | atagtgattcgaattgtgtgttgatttcat | 27 | 23 |
| | | | | | | agaataatacatatttatatacaaagcta | | |
| | | | | | | ggagactaaatatctactaaatatctac | | |
| | | | | | | taaatatctaatt | | |
| M28 | 1 | 20714289 | G | A | A | gagagactcgaacttcaaaccttgtgg | 28 | 40 |
| | | | | | | aagcaaaccatacacgtgaccatttga | | |
| | | | | | | actcttaggttctcctatgcttaatcacta | | |
| | | | | | | taagaatccgacaatt | | |
| M29 | 1 | 20714407 | C | A | C | ttttgcttggataatacatagatcccata | 29 | 32 |
| | | | | | | ttcacaaacaactagaatgaacaagg | | |
| | | | | | | gaaacacaaacatacaatttgattggat | | |
| | | | | | | gcagcttcatctatttt | | |
| M30 | 1 | 20714539 | G | C | G | tttattctttttgctgagttttagaactcaa | 30 | 32 |
| | | | | | | catgccactaaactaaattaactaatctt | | |
| | | | | | | cagtaaacataaaagttgtggctattaa | | |
| | | | | | | cccacttggtgg | | |
| M31 | 1 | 20715293 | G | A | A | gactctttatttcatcgatgagttgattag | 31 | 37 |
| | | | | | | catctctgagattacctgaagacaaata | | |
| | | | | | | cctacagtaacataattattgtaacagg | | |
| | | | | | | caagagtcagggca | | |
| M32 | 1 | 20716703 | G | C | C | gaatatcaacgcctagtaggtaagctg | 32 | 38 |
| | | | | | | acttacctttctcacacgcctaacattag | | |
| | | | | | | atttgcagtcaagtgttgttggtcaattt | | |
| | | | | | | atgaatgatcctaaa | | |
| M33 | 1 | 20722604 | A | C | C | ctagttggaccatgcatccaggttaac | 33 | 40 |
| | | | | | | attcacctagcgcatatggttcaattcat | | |
| | | | | | | caaattgtcacactagcaatggaaaaa | | |
| | | | | | | tgcttcttcagcatcaa | | |
| M34 | 1 | 20722924 | G | A | A | tcaccattacattaggaagtctcacatc | 34 | 42 |
| | | | | | | aaatcaacatcaaggtgcagcataac | | |
| | | | | | | attcagcacctgcaccacctaggtgct | | |
| | | | | | | taatcttgtttttctccag | | |
| M35 | 1 | 20724488 | C | T | T | tgaaacagtacacagatgcaacaattc | 35 | 30 |
| | | | | | | tcaataaaacagtaacaagtggtgaac | | |
| | | | | | | aaattagttattattgatagtaattgaaa | | |
| | | | | | | atcagacactagctcta | | |
| M36 | 1 | 20725130 | C | T | T | ccaattggatgcaagcacctgaatgaa | 36 | 40 |
| | | | | | | taatatccaaagcttcagaaaacctttg | | |
| | | | | | | ggcagatacatatctgcaataaagcat | | |
| | | | | | | gcatgcttcatcagtgac | | |
| M37 | 1 | 20766852 | A | G | G | tttactagtaagtatgtttttaacatccta | 37 | 28 |
| | | | | | | aatatgaatctctaaaacgatgaaactt | | |
| | | | | | | aaacacatataaagtatgagaaacctt | | |
| | | | | | | acattagttgcagcg | | |
| M38 | 1 | 20767720 | T | C | C | tccaataagcagatctagaatttatcaa | 38 | 31 |
| | | | | | | gtaaattccctaacttattaattcctcctt | | |
| | | | | | | gcaccactatagatttggaattgtactct | | |
| | | | | | | cgattatatagaa | | |
| M39 | 1 | 20767831 | G | A | A | ttccacgatataaatacgctatgagatt | 39 | 32 |
| | | | | | | atccatttgttataatcctaataatcagtg | | |
| | | | | | | atcctctatagatgatttacaccgagta | | |
| | | | | | | gggacaaatttatc | | |
| M40 | 1 | 20768240 | C | A | C | gattgagatcatttgatctaagatcaact | 40 | 30 |
| | | | | | | aggtgatattgaattgcatagatattac | | |
| | | | | | | ggtaaatttattatatctattccaagttca | | |
| | | | | | | atatcggtccctt | | |
| M41 | 1 | 20773638 | A | G | G | gcatgttgatggcatgacaagggagg | 41 | 40 |
| | | | | | | cgagcttgggggaaattgtgacaaatt | | |
| | | | | | | ttcattactttcaaaggagtgcctttttaa | | |
| | | | | | | ggttcaaaataagtact | | |
| M42 | 1 | 20875127 | A | G | G | tgcccccccgaaaactgaatggtgtg | 42 | 53 |
| | | | | | | ccatccgtcaacactgctcttgccacc | | |
| | | | | | | aactggcactaattcatctgagctgcct | | |
| | | | | | | gcatctgagattgagaggt | | |
| M43 | 1 | 21025217 | C | T | C | agcttcagaaacttcaggtttgatacttc | 43 | 38 |
| | | | | | | gtcaatcttacaagcaatggaaactgg | | |
| | | | | | | tttctctgcatgaattctaacactgctgc | | |
| | | | | | | tctgtaaagttgttt | | |
| M44 | 1 | 21167918 | A | C | A | atggagtttatggctgacaaatttgata | 44 | 37 |
| | | | | | | aaggttgcatcactcgattaaaaatggt | | |
| | | | | | | tgcttcaaccccctttgaacggataaca | | |
| | | | | | | tatacaaaagcagtag | | |
| M45 | 1 | 21179156 | C | T | C | atggcggaggtatgagagggattctgt | 45 | 53 |
| | | | | | | ccgggaaagcattggcatacttagag | | |
| | | | | | | cacgcgctcaaggctaaatcggggaa | | |
| | | | | | | tccagacgctagaatcgctga | | |
| M46 | 1 | 21179807 | T | A | T | tgcacaacaagcaggagtttccgttcg | 46 | 54 |
| | | | | | | tgcgtggggtggaggacctcttggtcc | | |
| | | | | | | tttccattggaacgggtcagctcttgga | | |
| | | | | | | agtgagtttcgagaacga | | |
| M47 | 1 | 21180216 | C | G | C | gtttatggtgcatgagacacagatggc | 47 | 39 |
| | | | | | | cagctgggaatggtcaaagaattttgt | | |
| | | | | | | cttttactagattttcccatgcatgacaat | | |
| | | | | | | ggtgtaatagctatta | | |
| M48 | 1 | 21199290 | A | G | G | ggatcatggcgaccacgggtgattaa | 48 | 40 |
| | | | | | | atctgtctcaattttatctctagtcactgt | | |
| | | | | | | atgctgcttcttctggaaaatatttaagg | | |
| | | | | | | ggaaaaaaaagcacc | | |
| M49 | 1 | 21200409 | G | T | T | taagctcatacttgaacgtcataaacag | 49 | 33 |
| | | | | | | ctatgagtaagtaaactgcctacagttc | | |
| | | | | | | ccagattagaaaatatgtaaattcaattt | | |
| | | | | | | gcaaattgataaggg | | |
| M50 | 1 | 21200988 | T | A | A | gtgaactagataactaccatcaatctta | 50 | 28 |
| | | | | | | tttggccatttcctcctatcaatcttatttg | | |
| | | | | | | atcatttcatctaaaagttctaaattatttt | | |
| | | | | | | gcgataatta | | |
| M51 | 1 | 21203266 | C | T | T | gagattctccataagttgtaacacgaa | 51 | 33 |
| | | | | | | aatgagttccaaaactattcccagctgc | | |
| | | | | | | ctttacttctgtatttcgacagcaaagat | | |
| | | | | | | attgtaattataattt | | |
| M52 | 1 | 21212936 | G | A | G | agtcctatttgtacaattttgaaaaatgc | 52 | 21 |
| | | | | | | aaggtccattttgttatttaacaaaacat | | |
| | | | | | | atgatctaattaatattttttacaaaatac | | |
| | | | | | | aaggtttaaaat | | |
| M53 | 1 | 21314684 | A | C | C | catccaacccaagcttactttaaccaat | 53 | 39 |
| | | | | | | gccctagaaaggacataacacttatcc | | |
| | | | | | | aaggtgcaagtaaacaacattgtaggt | | |
| | | | | | | tatcctatcagttaaacc | | |
| M54 | 1 | 21315426 | A | G | G | tgcggtttgtttagcacttctccaaacaa | 54 | 44 |
| | | | | | | ctgctcctcccccaagagtaaacacca | | |
| | | | | | | tcccagatgtagactttctgtcatcaag | | |
| | | | | | | acaagcctaaaaatct | | |
| M55 | 1 | 21327747 | G | A | A | ctaatatagattatttattatctttttaataa | 55 | 12 |
| | | | | | | tttgtttcatattgtattaaaatttataattg | | |
| | | | | | | taataattaatataattcaaaattcaatcc | | |
| | | | | | | aaacat | | |
| M56 | 1 | 21329106 | G | T | T | ttgtttcttagttaaaaacacagtcataa | 56 | 27 |
| | | | | | | ggtgagaaagcaagaacatttaattaa | | |
| | | | | | | tactagaagtaaaacaagacaatgtga | | |
| | | | | | | gcttatactagtttata | | |
| M57 | 1 | 21459680 | A | G | A | tagtgcagaaagattacctaccataag | 57 | 36 |
| | | | | | | aattttgttttgacgctgtaattctctacat | | |
| | | | | | | aatgattcaagtttatctcgaacagcaa | | |
| | | | | | | cagctacaacaggc | | |
| M58 | 1 | 21478041 | T | A | T | aagaaaagaaaaagtgtagttcagtgt | 58 | 29 |
| | | | | | | tgaggaaaaatctcacaaccaaaatta | | |
| | | | | | | ttttgtttcttaatgaccattaactaaaca | | |
| | | | | | | gcctatacttaaggta | | |
| M59 | 1 | 21478567 | A | T | A | tcatatcaggtgatgtcatgaatgtcac | 59 | 37 |
| | | | | | | aactggacctaatgatacagagagac | | |
| | | | | | | agcatgttgaaagtgataaaagctttac | | |
| | | | | | | tttctttatggtcgaaga | | |
| Marker_ | | | | | Marker_ | | SEQ | Right_ |
| Num | Chrom | Pos | Ref | Alt | Allele | Right_Seq | ID NO | GC |
| M01 | 1 | 19351704 | A | G | A | agatttttcttacaaaatccaaacccaat | 60 | 38 |
| | | | | | | ctctctcttttctctatacctctctccttga | | |
| | | | | | | atgcttctgtggtcgccataaattatgttt | | |
| | | | | | | tcgtggtgga | | |
| M02 | 1 | 19353247 | A | G | A | cattgtcttctgattccacactaacttcc | 61 | 41 |
| | | | | | | atctgtgaaccttctccgaaagcttcgtt | | |
| | | | | | | ccgtactccatttccacacttacagtgt | | |
| | | | | | | ctttgtttcttatt | | |
| M03 | 1 | 19402679 | T | A | A | tcatcatcatcagcatcatctacatcatc | 62 | 31 |
| | | | | | | atcttcctctggttcatcttctttttcttctt | | |
| | | | | | | ctaacatttgtaataatcaagaattaaaa | | |
| | | | | | | cagttgaaga | | |
| M04 | 1 | 19412546 | G | A | G | aaaggaaaacatcagtatattaagaat | 63 | 29 |
| | | | | | | atgtaaatataaaactgtaatgagatgc | | |
| | | | | | | ttggctataattcaagttttctcatttgcat | | |
| | | | | | | aagcgtgcggatat | | |
| M05 | 1 | 19413329 | A | G | A | agtaaaagaatgttagagaaaagcatt | 64 | 33 |
| | | | | | | cagcacgatatcaaataatgaggagg | | |
| | | | | | | accatagcagacaataacgaaacctat | | |
| | | | | | | tcttacaagtatagacaaat | | |
| M06 | 1 | 19682959 | G | T | T | gccctttgaggataacaattcttagtaat | 65 | 36 |
| | | | | | | ttttttaagtttctctataatctgcataattc | | |
| | | | | | | tatttgccaccggcactgggtgcttcca | | |
| | | | | | | ctttgatatac | | |
| M07 | 1 | 19687541 | C | T | C | agaggagttatagctccctactattcga | 66 | 40 |
| | | | | | | gaatgacgagctgtttgtgagctatac | | |
| | | | | | | cttgcctaagatagattatgaggaacta | | |
| | | | | | | gacccaatgactaataa | | |
| M08 | 1 | 19692966 | G | C | C | aaaaagagatggactaggagctggg | 67 | 38 |
| | | | | | | cctggtcaaaatccccaggtctgataa | | |
| | | | | | | taataacatgaagaggttaatggtgcc | | |
| | | | | | | attttattttgcttttagtat | | |
| M09 | 1 | 19696755 | G | T | T | tatagtgggctcgccatggaagactgc | 68 | 42 |
| | | | | | | ttagatgtatggctccaaaacctatgaa | | |
| | | | | | | ccgaagtcgttcttttctcatttttgcagt | | |
| | | | | | | atatatggctagtca | | |
| M10 | 1 | 19713019 | T | C | C | atatttatataatgtatgtataaacctata | 69 | 21 |
| | | | | | | attacaaagatataaatacacagattaa | | |
| | | | | | | gaatcacaccttatgtcacaactataca | | |
| | | | | | | cattaaataaatct | | |
| M11 | 1 | 19713824 | G | A | A | ttgtagcttagcttctgtataccatcatc | 70 | 25 |
| | | | | | | acatacgatccacttcattaaaaaattat | | |
| | | | | | | taactataacaaacaaatatttatcaga | | |
| | | | | | | aaataaaaatcttg | | |
| M12 | 1 | 19717871 | G | A | A | agcaagtgaaggaggggtcccacat | 71 | 37 |
| | | | | | | gtactatataagggattatggggggaa | | |
| | | | | | | cccttttcactttttactttttacttctctca | | |
| | | | | | | ctataaattgaaattt | | |
| M13 | 1 | 19718025 | T | C | C | taggatttttgcgccgtttcgatgatcta | 72 | 39 |
| | | | | | | agcttcccacattcttcctcaacaatttc | | |
| | | | | | | ttgtagtgggtaagctttctattcccgtt | | |
| | | | | | | agttcacttaatt | | |
| M14 | 1 | 19807569 | G | A | A | gagaaagcctctcgaagagctattttc | 73 | 34 |
| | | | | | | agtaattctttggtgattaatagaaagc | | |
| | | | | | | attcctgtgggaattcattactgtagttg | | |
| | | | | | | tttgtgttgatatata | | |
| M15 | 1 | 19812569 | T | C | C | aagtgttcaatgctggtaagatctttgat | 74 | 34 |
| | | | | | | aatgcttacaaaaatagcactgtcttct | | |
| | | | | | | ataactccaacaaagtaaagttccaaa | | |
| | | | | | | agtagcttcagagtac | | |
| M16 | 1 | 19812701 | T | C | C | gtatctgagtcatcccctgattttccag | 75 | 44 |
| | | | | | | gaaagaagactttcaaaagcccttgaa | | |
| | | | | | | caaggctcggggagaagtctttgtatc | | |
| | | | | | | tttgatgaagcaaagagc | | |
| M17 | 1 | 19950755 | T | C | T | ttaatagtaaaatgagaagttaaatgta | 76 | 23 |
| | | | | | | aaaggatgaactaaatactcgcatatg | | |
| | | | | | | tcaaaacaaaaacaccaaaaataacta | | |
| | | | | | | agtataaaattagttcta | | |
| M18 | 1 | 19958734 | C | G | G | agggcgatgtcgcgaggtaccgagat | 77 | 53 |
| | | | | | | ctcgcggtgttagtagcagagggtgc | | |
| | | | | | | ctcagtgatgatgggacctccttccttt | | |
| | | | | | | gtatcatacatttaccttcg | | |
| M19 | 1 | 19988215 | G | A | G | atttcttgacctagcttagattttgacata | 78 | 34 |
| | | | | | | gaaccattcttgaggatactacagtgg | | |
| | | | | | | gttacttagtttgtagagtatgtttatgtg | | |
| | | | | | | taccttctaaaga | | |
| M20 | 1 | 19988827 | G | T | T | tgagtatatttcctttattttggatttaaaa | 79 | 20 |
| | | | | | | taatacttttttctctagtatctttttgtaatt | | |
| | | | | | | tatacttttaactaatacatttattgtgtgt | | |
| | | | | | | gtttg | | |
| M21 | 1 | 19988955 | A | T | A | catgattctaggagtatggtctttaagtg | 80 | 38 |
| | | | | | | tttatcgaaaggtgccgttgactttttag | | |
| | | | | | | tgaaacctattcgaaagaatgagctga | | |
| | | | | | | aaaacctttggcaac | | |
| M22 | 1 | 19994256 | G | T | G | tgtttgccttctagaattcataaaagacc | 81 | 48 |
| | | | | | | tacagggcggtagtttccaaattctcga | | |
| | | | | | | cctccttcgagagctcttcttccctcgtc | | |
| | | | | | | tgcctggccttaac | | |
| M23 | 1 | 20011591 | A | T | A | acataacaaaaacactatacttccaag | 82 | 33 |
| | | | | | | ctttataatgtgaccatcatggtaaacc | | |
| | | | | | | agcaaacgcaccatcatgttatttacaa | | |
| | | | | | | atgaagctaaccatatt | | |
| M24 | 1 | 20015613 | G | A | G | tgatgtttcacaagtgaaaggccacag | 83 | 36 |
| | | | | | | gggttaaataaaaaagaacaaagaaa | | |
| | | | | | | acaagcattacctgagattctatcatttc | | |
| | | | | | | ctctgagtaatagccatc | | |
| M25 | 1 | 20016226 | G | A | G | tccagtgctgggtgacctgggaatgtt | 84 | 45 |
| | | | | | | cgaggcaaatcctgcaaatggaatca | | |
| | | | | | | caaactagaagttgggaaaatgcaaa | | |
| | | | | | | gctgctacttataatgagcac | | |
| M26 | 1 | 20030132 | C | T | C | agcctctctaagtatggccaactaaaa | 85 | 47 |
| | | | | | | gtcttctcctaagtccacatgaggctca | | |
| | | | | | | ccaattgttggttcgccaattcctggcc | | |
| | | | | | | ctatttccccagcataa | | |
| M27 | 1 | 20712699 | A | C | A | ttttacagctaatatacatctaatatctaa | 86 | 27 |
| | | | | | | cactaagaaatatggggaacggagaa | | |
| | | | | | | atataagttatttcataaggtaaatccatt | | |
| | | | | | | aacaatacattgac | | |
| M28 | 1 | 20714289 | G | A | A | gacccaatggtgcataattttgcttgga | 87 | 34 |
| | | | | | | taatacatagatcccatattcacaaaca | | |
| | | | | | | actagaatgaacaagggaaacacaaa | | |
| | | | | | | catacaatttgattggat | | |
| M29 | 1 | 20714407 | C | A | C | acctttcaatttctttagactttcatttgttt | 88 | 27 |
| | | | | | | ttattctttttgctgagttttagaactcaac | | |
| | | | | | | atgccactaaactaaattaactaatcttc | | |
| | | | | | | agtaaaca | | |
| M30 | 1 | 20714539 | G | C | G | cctgagaaattaaaccatagtgataat | 89 | 39 |
| | | | | | | ggtgggacagcccctgagttctgtcat | | |
| | | | | | | aagtttagtactcagatcgcattgtcaat | | |
| | | | | | | atttttcaaagccacta | | |
| M31 | 1 | 20715293 | G | A | A | tctatcagaagaagaaaggagagaga | 90 | 35 |
| | | | | | | gaaaaatagatggaatgctgattaggg | | |
| | | | | | | agtttgaaattggagatgttgttagcctt | | |
| | | | | | | taaataatgggagaagta | | |
| M32 | 1 | 20716703 | G | C | C | aagttcaccaacaagttgtttacagattt | 91 | 38 |
| | | | | | | acaaggtatctgaagatgacacctggt | | |
| | | | | | | aaagggtcgtacttcaagaagggaac | | |
| | | | | | | taacaagaacattgagat | | |
| M33 | 1 | 20722604 | A | C | C | aataccctaacttcgcataacctgaga | 92 | 52 |
| | | | | | | ggttgcaccaagtgaactgacaccag | | |
| | | | | | | gtgcccaagtgacaccatgcgccaag | | |
| | | | | | | tgaattgacaccaggtgccca | | |
| M34 | 1 | 20722924 | G | A | A | aaatcatgcacaacttaagcatttgact | 93 | 35 |
| | | | | | | tagtgatgcaccaatatcacattagcaa | | |
| | | | | | | tccaaaatatgtcaaaatcatgtatttct | | |
| | | | | | | acgcccaggtgaaca | | |
| M35 | 1 | 20724488 | C | T | T | agggaaaattagagtcgaagctctcc | 94 | 38 |
| | | | | | | aagaatgctaagaattactaagttcttg | | |
| | | | | | | cataaccctcactcagttcacatacatt | | |
| | | | | | | ctataagagcaactcaac | | |
| M36 | 1 | 20725130 | C | T | T | tgaattggtgataaagtgtaacaagtta | 95 | 35 |
| | | | | | | catgctaggtgaaaataacatatgacg | | |
| | | | | | | gataccaaatgggcaaagtttgagttg | | |
| | | | | | | atacaaatgtaggttcgt | | |
| M37 | 1 | 20766852 | A | G | G | aattaaatgactccttctactcagatctc | 96 | 40 |
| | | | | | | taaccattgttcctttctgtcgcagagta | | |
| | | | | | | tgatcaagatttgagcccgacactcctt | | |
| | | | | | | cagttgtttggatt | | |
| M38 | 1 | 20767720 | T | C | C | gctctatatgttccacgatataaatacg | 97 | 34 |
| | | | | | | ctatgagattatccatttgttataatccta | | |
| | | | | | | ataatcagtgatcctctatagatgattta | | |
| | | | | | | caccgagtaggga | | |
| M39 | 1 | 20767831 | G | A | A | ttacactcttcaatgtattttatccttaaaa | 98 | 24 |
| | | | | | | caattagctacatataaatgatatttaag | | |
| | | | | | | tgatctaatataatcactgaaatgagca | | |
| | | | | | | ctcaatcatata | | |
| M40 | 1 | 20768240 | C | A | C | cgatgcatacttatacacccaacccaa | 99 | 39 |
| | | | | | | gtttactttaaccaatgccctggaaatg | | |
| | | | | | | acataacacttatgcacggtgcaagta | | |
| | | | | | | aactacactgtagattat | | |
| M41 | 1 | 20773638 | A | G | G | tcatggaaatttgaatttcgaaaagtaa | 100 | 31 |
| | | | | | | ctaaatgtgggacttagcgtaattggtt | | |
| | | | | | | gggtgattttactacacgtgtctttatttc | | |
| | | | | | | cttaagattatttt | | |
| M42 | 1 | 20875127 | A | G | G | gagaagaatcagagagagcataaaat | 101 | 34 |
| | | | | | | gacactaaagaatgtagtaatgaggct | | |
| | | | | | | tttgtcaaacatcagaagatgattcgaa | | |
| | | | | | | aatggtgaacacaaaaaca | | |
| M43 | 1 | 21025217 | C | T | C | acttgttttactactcaccaattagttctt | 102 | 42 |
| | | | | | | cccgcaatgtttgacggtggcccccct | | |
| | | | | | | gtatagcggttcaagaattccagtttcg | | |
| | | | | | | ggttttaagtaaatt | | |
| M44 | 1 | 21167918 | A | C | A | gcttttagaagaggctgtgaagaatgg | 103 | 38 |
| | | | | | | taagcagtttgagaacaaggtagagtg | | |
| | | | | | | gggaattgatttggcatctgaacatga | | |
| | | | | | | aaggtatgcaaattttatt | | |
| M45 | 1 | 21179156 | C | T | C | tacttcgacgtggcagcgggggctgg | 104 | 54 |
| | | | | | | tgttggaggaattttcacggctatgctct | | |
| | | | | | | ttgctacgagtgaccagagccgccca | | |
| | | | | | | atatccaaagccgatgata | | |
| M46 | 1 | 21179807 | T | A | T | caagtcaaaaagtggaaggccaagg | 105 | 58 |
| | | | | | | attgggcacgtcccactgcccgtattg | | |
| | | | | | | ctagcgacggctccgctgacctagttg | | |
| | | | | | | atcaggccgtctccatggcct | | |
| M47 | 1 | 21180216 | C | ? | C | aatactagatagaattgaaggtgatatt | 106 | 41 |
| | | | | | | gatattgggttggggatgggctaacgt | | |
| | | | | | | gcgccggagttttgtgtttatgcaattaa | | |
| | | | | | | atgtcgggtgtagttg | | |
| M48 | 1 | 21199290 | A | G | G | aaaaaaacagatttaggcaacacaact | 107 | 32 |
| | | | | | | cttttagattatcaaccaactccacactc | | |
| | | | | | | aaactacttcgcgaaaaaagaaatatc | | |
| | | | | | | aagcagaggattatttt | | |
| M49 | 1 | 21200409 | G | T | T | aaatggaaaatggaagaaagaagcct | 108 | 44 |
| | | | | | | tacacgtggacaattcttcacaacacat | | |
| | | | | | | gtaacaacgccgccaatggaatcccc | | |
| | | | | | | tctcacccggatagtatcta | | |
| M50 | 1 | 21200988 | T | A | A | atactaaacttcacgatcagcattcaaa | 109 | 44 |
| | | | | | | atggtacctgatcaagtgtcaaggcct | | |
| | | | | | | catgatcaaccaaccccagcggaagc | | |
| | | | | | | tcaacattgtgtacttgtg | | |
| M51 | 1 | 21203266 | C | T | T | atgccaattcagttcaagtcattcagca | 110 | 32 |
| | | | | | | aagccaatggtatttactttagatgtaat | | |
| | | | | | | catttactttcaagtttgcaaataaagca | | |
| | | | | | | cacaagaacactta | | |
| M52 | 1 | 21212936 | G | A | G | gtatttattgttattgttattgaatattagtt | 111 | 21 |
| | | | | | | gtgctcagtacttttcataggtgattctat | | |
| | | | | | | tagtgatattctttaaaagttatttttttaa | | |
| | | | | | | cattata | | |
| M53 | 1 | 21314684 | A | C | C | tgtgtactgataaattataggaatacatt | 112 | 28 |
| | | | | | | taatcacataatcttaaatactttccact | | |
| | | | | | | gtgctgacgacacaataaacaagaat | | |
| | | | | | | atcaatgtgataagaa | | |
| M54 | 1 | 21315426 | A | G | G | agtcggtatagcctacgggatttaaag | 113 | 40 |
| | | | | | | caccactcttgtagactaacacataatg | | |
| | | | | | | ccttgtacttttcaagtacttcagaatat | | |
| | | | | | | gcttaactgcagtcca | | |
| M55 | 1 | 21327747 | G | A | A | gtattttacttatttaatgcaataggtata | 114 | 25 |
| | | | | | | ccaagcatgccctaagaaaatcaacta | | |
| | | | | | | caacttatttaaattgttaaaactatattat | | |
| | | | | | | tcaactaacatc | | |
| M56 | 1 | 21329106 | G | T | T | actaaagcactctttcaacttttatacaa | 115 | 23 |
| | | | | | | taaaattcattaaaagaagaagtttgca | | |
| | | | | | | tttcttaagaattacataattgctacttaa | | |
| | | | | | | gaattacatatag | | |
| M57 | 1 | 21459680 | A | G | A | gggtgagaccaaaaaatttatcaatta | 116 | 24 |
| | | | | | | gacaaatacgttgctcatattcaaacat | | |
| | | | | | | actataaaacatggaaaatttaatgtga | | |
| | | | | | | aatttattttatgaaaa | | |
| M58 | 1 | 21478041 | T | A | T | gcagtcttataaatctcaaaatgccaaa | 117 | 26 |
| | | | | | | atctctatttatgatgtgatagaataaca | | |
| | | | | | | taaatattcctcattcatcaacatcataa | | |
| | | | | | | atcacaaatttttg | | |
| M59 | 1 | 21478567 | A | T | A | attggtccatagtagacaatcccagag | 118 | 42 |
| | | | | | | tctttttcattttccccatcttctgctacttt | | |
| | | | | | | tcttgggccaatactgagctttccattgt | | |
| | | | | | | cttcagctgtag | | |

### Linkage Drag

When plant breeding introduces a desired gene (“target gene”) from a donor parent to improve a cultivar for a specific trait, other genes closely linked to the target gene are also typically carried from the donor parent to the recipient cultivar. The undesired alleles of non-target genes from the donor parent, because of their close linkage with the target gene, often persist even after multiple backcrosses. The persistent non-target genes often reduce the fitness or desirability of the backcross progeny—a phenomenon known as linkage drag. Molecular makers offer a tool in which the amount of donor DNA can be monitored during each backcross generation, in order to reduce linkage drag.

It is well known that efforts to introgress the AF trait into other cultivars of Cannabis results in progeny that are not as phenotypically desirable as the original photoperiod parent. This can be attributed to linkage drag. Accordingly, the markers of the present invention can be used to monitor and minimize linkage drag as plants are crossed and backcrossed in efforts to introgress AF into Value Phenotype recipient plants.

Inheritance patterns from crosses of AF and photoperiod parents indicate that AF is determined by a recessive allele of a single gene. The markers of the present invention define a region of chromosome 1 in which this single AF locus resides. The region defined by these markers comprises 98 transcripts, according to Cannabis sativa cs10 RefSeq assembly accession: GCF_900626175.2 (Assembly [Internet]. Bethesda (MD): National Library of Medicine (US), National Center for Biotechnology Information; 2012-2022 Jan. 24. Accession No. GCF_900626175.2, cs10; Available from: www<dot>ncbi<dot>nlm<dot>nih<dot>gov<slash>assembly<slash>GCF_900626175.2). Table 2 lists genes and positions within the segment of the chromosome defined by the markers. Thus, given that only one gene from Table 2 controls the AF trait, many or all of the other genes listed in Table 2 contribute to linkage drag, to some degree. The invention includes a breeding protocol capable of introgressing the AF gene into a Value Phenotype recipient parent, while leaving most or all of the other genes listed in Table 2 behind, will result in an improved AF Value Phenotype cultivar.

TABLE 2

| seqname | Cs10_Chr | Start_Pos | End_Pos | Gene | Product | Marker_Num |
|---|---|---|---|---|---|---|
| NC_044371.1 | 1 | 19342709 | 19347249 | gene = | product = protein- | |
| | | | | LOC115707983 | tyrosine-phosphatase | |
| | | | | | MKP1, transcript variant | |
| | | | | | X1 | |
| NC_044371.1 | 1 | 19342709 | 19347249 | gene = | product = protein- | |
| | | | | LOC115707983 | tyrosine-phosphatase | |
| | | | | | MKP1, transcript variant | |
| | | | | | X2 | |
| NC_044371.1 | 1 | 19347249 | 19354466 | Intergenic | — | M01, M02 |
| NC_044371.1 | 1 | 19354466 | 19362100 | gene = | product = beta- | |
| | | | | LOC115707986 | hexosaminidase 1 | |
| NC_044371.1 | 1 | 19368217 | 19380104 | gene = | product = probable DNA | |
| | | | | LOC115707984 | double-strand break | |
| | | | | | repair Rad50 ATPase | |
| NC_044371.1 | 1 | 19381034 | 19403194 | gene = | product = probable | M03 |
| | | | | LOC115707987 | membrane-associated | |
| | | | | | kinase regulator 4 | |
| NC_044371.1 | 1 | 19411191 | 19415240 | gene = | product = ankyrin repeat- | M04, M05 |
| | | | | LOC115707985 | containing protein ITN1 | |
| NC_044371.1 | 1 | 19586800 | 19590447 | gene = | product = uncharacterized | |
| | | | | LOC115706681 | LOC115706681, | |
| | | | | | transcript variant X2 | |
| NC_044371.1 | 1 | 19586801 | 19591181 | gene = | product = uncharacterized | |
| | | | | LOC115706681 | LOC115706681, | |
| | | | | | transcript variant X1 | |
| NC_044371.1 | 1 | 19623001 | 19626945 | gene = | product = protein NRT1/ | |
| | | | | LOC115708189 | PTR FAMILY 2.7 | |
| NC_044371.1 | 1 | 19670607 | 19672347 | gene = | product = uncharacterized | |
| | | | | LOC115703863 | LOC115703863 | |
| NC_044371.1 | 1 | 19675794 | 19679721 | gene = | product = protein NRT1/ | |
| | | | | LOC115706683 | PTR FAMILY 2.7-like | |
| NC_044371.1 | 1 | 19679721 | 19691506 | Intergenic | — | M06, M07 |
| NC_044371.1 | 1 | 19691506 | 19696923 | gene = | product = nuclear | M08, M09 |
| | | | | LOC115706176 | transcription factor Y | |
| | | | | | subunit B-1, transcript | |
| | | | | | variant X2 | |
| NC_044371.1 | 1 | 19691506 | 19696923 | gene = | product = nuclear | M08, M09 |
| | | | | LOC115706176 | transcription factor Y | |
| | | | | | subunit B-1, transcript | |
| | | | | | variant X4 | |
| NC_044371.1 | 1 | 19691507 | 19696923 | gene = | product = nuclear | M08, M09 |
| | | | | LOC115706176 | transcription factor Y | |
| | | | | | subunit B-1, transcript | |
| | | | | | variant X1 | |
| NC_044371.1 | 1 | 19691507 | 19696923 | gene = | product = nuclear | M08, M09 |
| | | | | LOC115706176 | transcription factor Y | |
| | | | | | subunit B-1, transcript | |
| | | | | | variant X3 | |
| NC_044371.1 | 1 | 19691507 | 19696923 | gene = | product = nuclear | M08, M09 |
| | | | | LOC115706176 | transcription factor Y | |
| | | | | | subunit B-1, transcript | |
| | | | | | variant X5 | |
| NC_044371.1 | 1 | 19712612 | 19715469 | gene = | product = probable RNA- | M10, M11 |
| | | | | LOC115704691 | binding protein ARP1 | |
| NC_044371.1 | 1 | 19715469 | 19726723 | Intergenic | — | M12, M13 |
| NC_044371.1 | 1 | 19726723 | 19728921 | gene = | product = floral homeotic | |
| | | | | LOC115708151 | protein APETALA 2, | |
| | | | | | transcript variant X1 | |
| NC_044371.1 | 1 | 19726723 | 19728918 | gene = | product = floral homeotic | |
| | | | | LOC115708151 | protein APETALA 2, | |
| | | | | | transcript variant X2 | |
| NC_044371.1 | 1 | 19778639 | 19780198 | gene = | product = uncharacterized | |
| | | | | LOC115703865 | LOC115703865 | |
| NC_044371.1 | 1 | 19782063 | 19783840 | gene = | product = uncharacterized | |
| | | | | LOC115703866 | LOC115703866 | |
| NC_044371.1 | 1 | 19802609 | 19815150 | gene = | product = regulator of | M14, M15, |
| | | | | LOC115706264 | nonsense transcripts | M16 |
| | | | | | UPF2 | |
| NC_044371.1 | 1 | 19822088 | 19823007 | gene = | product = uncharacterized | |
| | | | | LOC115703868 | LOC115703868 | |
| NC_044371.1 | 1 | 19826131 | 19827204 | gene = | product = uncharacterized | |
| | | | | LOC115703869 | LOC115703869 | |
| NC_044371.1 | 1 | 19843513 | 19847204 | gene = | product = zinc finger | |
| | | | | LOC115706080 | CCCH domain- | |
| | | | | | containing protein 11 | |
| NC_044371.1 | 1 | 19849983 | 19850489 | gene = | product = uncharacterized | |
| | | | | LOC115703870 | LOC115703870 | |
| NC_044371.1 | 1 | 19860264 | 19863668 | gene = | product = protein | |
| | | | | LOC115703871 | TONNEAU 1a-like | |
| NC_044371.1 | 1 | 19863668 | 19985933 | Intergenic | — | M17, M18 |
| NC_044371.1 | 1 | 19985933 | 19992033 | gene = | product = two-component | M19, M20, |
| | | | | LOC115705128 | response regulator-like | M21 |
| | | | | | PRR37 | |
| NC_044371.1 | 1 | 19992033 | 20010950 | Intergenic | — | M22 |
| NC_044371.1 | 1 | 20010950 | 20018438 | gene = | product = TBC1 domain | M23, M24, |
| | | | | LOC115704703 | family member 8B | M25 |
| NC_044371.1 | 1 | 20018438 | 20032520 | Intergenic | — | M26 |
| NC_044371.1 | 1 | 20032520 | 20036951 | gene = | product = CDP- | |
| | | | | LOC115705441 | diacylglycerol--glycerol- | |
| | | | | | 3-phosphate 3- | |
| | | | | | phosphatidyltransferase | |
| | | | | | 2 | |
| NC_044371.1 | 1 | 20574051 | 20576803 | gene = | product = uncharacterized | |
| | | | | LOC115705487 | LOC115705487 | |
| NC_044371.1 | 1 | 20595436 | 20599191 | gene = | product = uncharacterized | |
| | | | | LOC115703873 | LOC115703873 | |
| NC_044371.1 | 1 | 20615998 | 20619859 | gene = | product = WD repeat- | |
| | | | | LOC115708215 | containing protein | |
| | | | | | WRAP73, transcript | |
| | | | | | variant X1 | |
| NC_044371.1 | 1 | 20615998 | 20619859 | gene = | product = WD repeat- | |
| | | | | LOC115708215 | containing protein | |
| | | | | | WRAP73, transcript | |
| | | | | | variant X2 | |
| NC_044371.1 | 1 | 20640845 | 20644771 | gene = | product = protein IQ- | |
| | | | | LOC115706652 | DOMAIN 1-like | |
| NC_044371.1 | 1 | 20653407 | 20659939 | gene = | product = calcium-binding | |
| | | | | LOC115705663 | mitochondrial carrier | |
| | | | | | protein SCaMC-1-like | |
| NC_044371.1 | 1 | 20664332 | 20664739 | gene = | product = low | |
| | | | | LOC115707338 | temperature-induced | |
| | | | | | protein lt101.2 | |
| NC_044371.1 | 1 | 20667500 | 20669307 | gene = | product = LOB domain- | |
| | | | | LOC115704698 | containing protein 1 | |
| NC_044371.1 | 1 | 20696892 | 20698904 | gene = | product = uncharacterized | |
| | | | | LOC115708282 | LOC115708282 | |
| NC_044371.1 | 1 | 20698904 | 20713556 | Intergenic | — | M27 |
| NC_044371.1 | 1 | 20713556 | 20727975 | gene = | product = Golgi to ER | M28, M29, |
| | | | | LOC115705207 | traffic protein 4 homolog | M30, M31, |
| | | | | | | M32, M33, |
| | | | | | | M34, M35, |
| | | | | | | M36 |
| NC_044371.1 | 1 | 20735420 | 20738200 | gene = | product = uncharacterized | |
| | | | | LOC115703875 | LOC115703875 | |
| NC_044371.1 | 1 | 20760091 | 20762582 | gene = | product = uncharacterized | |
| | | | | LOC115703876 | LOC115703876 | |
| NC_044371.1 | 1 | 20762582 | 20775753 | Intergenic | — | M37, M38, |
| | | | | | | M39, M40, |
| | | | | | | M41 |
| NC_044371.1 | 1 | 20775753 | 20778199 | gene = | product = uncharacterized | |
| | | | | LOC115703877 | LOC115703877 | |
| NC_044371.1 | 1 | 20790932 | 20795500 | gene = | product = uncharacterized | |
| | | | | LOC115706745 | LOC115706745, | |
| | | | | | transcript variant X1 | |
| NC_044371.1 | 1 | 20790932 | 20795500 | gene = | product = uncharacterized | |
| | | | | LOC115706745 | LOC115706745, | |
| | | | | | transcript variant X2 | |
| NC_044371.1 | 1 | 20816258 | 20818673 | gene = | product = protein FAR1- | |
| | | | | LOC115703878 | RELATED SEQUENCE | |
| | | | | | 5-like | |
| NC_044371.1 | 1 | 20830310 | 20833207 | gene = | product = uncharacterized | |
| | | | | LOC115703879 | LOC115703879 | |
| NC_044371.1 | 1 | 20852425 | 20858895 | gene = | product = pre-rRNA- | |
| | | | | LOC115706767 | processing protein TSR1 | |
| | | | | | homolog | |
| NC_044371.1 | 1 | 20861533 | 20868270 | gene = | product = | |
| | | | | LOC115706769 | phosphoglucomutase | |
| NC_044371.1 | 1 | 20874609 | 20881142 | gene = | product = endoplasmic | M42 |
| | | | | LOC115706728 | reticulum | |
| | | | | | metallopeptidase 1-like | |
| NC_044371.1 | 1 | 20892287 | 20897961 | gene = | product = DNA | |
| | | | | LOC115706762 | polymerase epsilon | |
| | | | | | subunit 3-like | |
| NC_044371.1 | 1 | 20898688 | 20900527 | gene = | product = uncharacterized | |
| | | | | LOC115703880 | LOC115703880 | |
| NC_044371.1 | 1 | 20901023 | 20905614 | gene = | product = 3- | |
| | | | | LOC115706743 | hydroxyisobutyryl-CoA | |
| | | | | | hydrolase-like protein 2, | |
| | | | | | mitochondrial | |
| NC_044371.1 | 1 | 20957532 | 20960672 | gene = | product = bifunctional | |
| | | | | LOC115703881 | dihydrofolate reductase- | |
| | | | | | thymidylate synthase 1- | |
| | | | | | like | |
| NC_044371.1 | 1 | 20962955 | 20970736 | gene = | product = diaminopimelate | |
| | | | | LOC115706734 | decarboxylase 2, | |
| | | | | | chloroplastic | |
| NC_044371.1 | 1 | 20996324 | 20998378 | gene = | product = uncharacterized | |
| | | | | LOC115703882 | LOC115703882 | |
| NC_044371.1 | 1 | 20998925 | 20999638 | gene = | product = protein PXR1- | |
| | | | | LOC115706761 | like | |
| NC_044371.1 | 1 | 21021481 | 21025532 | gene = | product = mRNA- | M43 |
| | | | | LOC115706748 | decapping enzyme | |
| | | | | | subunit 2 | |
| NC_044371.1 | 1 | 21030259 | 21033631 | gene = | product = DNA | |
| | | | | LOC115706763 | polymerase epsilon | |
| | | | | | subunit 3 | |
| NC_044371.1 | 1 | 21044054 | 21048463 | gene = | product = 3- | |
| | | | | LOC115706744 | hydroxyisobutyryl-CoA | |
| | | | | | hydrolase-like protein 2, | |
| | | | | | mitochondrial | |
| NC_044371.1 | 1 | 21082797 | 21086224 | gene = | product = aquaporin PIP2- | |
| | | | | LOC115706754 | 2 | |
| NC_044371.1 | 1 | 21100198 | 21104415 | gene = | product = bifunctional | |
| | | | | LOC115706733 | dihydrofolate reductase- | |
| | | | | | thymidylate synthase, | |
| | | | | | transcript variant X5 | |
| NC_044371.1 | 1 | 21105580 | 21109352 | gene = | product = diaminopimelate | |
| | | | | LOC115706735 | decarboxylase 2, | |
| | | | | | chloroplastic-like | |
| NC_044371.1 | 1 | 21134331 | 21139980 | gene = | product = phosphatidylinositol/ | |
| | | | | LOC115703883 | phosphatidylcholine | |
| | | | | | transfer protein SFH3- | |
| | | | | | like | |
| NC_044371.1 | 1 | 21142406 | 21146635 | gene = | product = trafficking | |
| | | | | LOC115706760 | protein particle complex | |
| | | | | | subunit 1, transcript | |
| | | | | | variant X1 | |
| NC_044371.1 | 1 | 21142554 | 21144446 | gene = | product = trafficking | |
| | | | | LOC115706760 | protein particle complex | |
| | | | | | subunit 1, transcript | |
| | | | | | variant X2 | |
| NC_044371.1 | 1 | 21142554 | 21144432 | gene = | product = trafficking | |
| | | | | LOC115706760 | protein particle complex | |
| | | | | | subunit 1, transcript | |
| | | | | | variant X3 | |
| NC_044371.1 | 1 | 21147123 | 21147770 | gene = | product = uncharacterized | |
| | | | | LOC115703884 | LOC115703884 | |
| NC_044371.1 | 1 | 21152489 | 21155502 | gene = | product = uncharacterized | |
| | | | | LOC115706764 | LOC115706764, | |
| | | | | | transcript variant X1 | |
| NC_044371.1 | 1 | 21152489 | 21155502 | gene = | product = uncharacterized | |
| | | | | LOC115706764 | LOC115706764, | |
| | | | | | transcript variant X2 | |
| NC_044371.1 | 1 | 21152489 | 21155502 | gene = | product = uncharacterized | |
| | | | | LOC115706764 | LOC115706764, | |
| | | | | | transcript variant X4 | |
| NC_044371.1 | 1 | 21152581 | 21155502 | gene = | product = uncharacterized | |
| | | | | LOC115706764 | LOC115706764, | |
| | | | | | transcript variant X3 | |
| NC_044371.1 | 1 | 21152591 | 21155502 | gene = | product = uncharacterized | |
| | | | | LOC115706764 | LOC115706764, | |
| | | | | | transcript variant X5 | |
| NC_044371.1 | 1 | 21155973 | 21157289 | gene = | product = caffeoylshikimate | |
| | | | | LOC115706749 | esterase | |
| NC_044371.1 | 1 | 21157426 | 21161133 | gene = | product = WPP domain- | |
| | | | | LOC115706727 | associated protein | |
| NC_044371.1 | 1 | 21165867 | 21168970 | gene = | product = asparagine-- | M44 |
| | | | | LOC115706732 | tRNA ligase, | |
| | | | | | cytoplasmic 1 | |
| NC_044371.1 | 1 | 21171737 | 21172419 | gene = | product = sulfated surface | |
| | | | | LOC115703886 | glycoprotein 185 | |
| NC_044371.1 | 1 | 21178192 | 21184371 | gene = | product = patatin-like | M45, M46, |
| | | | | LOC115706736 | protein 6 | M47 |
| NC_044371.1 | 1 | 21198455 | 21204613 | gene = | product = chorismate | M48, M49, |
| | | | | LOC115706741 | synthase, chloroplastic | M50, M51 |
| NC_044371.1 | 1 | 21204613 | 21270041 | Intergenic | — | M52 |
| NC_044371.1 | 1 | 21270041 | 21271053 | gene = | product = uncharacterized | |
| | | | | LOC115703887 | LOC115703887 | |
| NC_044371.1 | 1 | 21271053 | 21328132 | Intergenic | — | M53, M54, |
| | | | | | | M55 |
| NC_044371.1 | 1 | 21328132 | 21332291 | gene = | product = protein IQ- | M56 |
| | | | | LOC115706740 | DOMAIN 1 | |
| NC_044371.1 | 1 | 21371455 | 21375371 | gene = | product = WD repeat- | |
| | | | | LOC115706772 | containing protein | |
| | | | | | WRAP73-like | |
| NC_044371.1 | 1 | 21381497 | 21382484 | gene = | product = uncharacterized | |
| | | | | LOC115703888 | LOC115703888 | |
| NC_044371.1 | 1 | 21416708 | 21419512 | gene = | product = uncharacterized | |
| | | | | LOC115706747 | LOC115706747 | |
| NC_044371.1 | 1 | 21433547 | 21437041 | gene = | product = 18S rRNA | |
| | | | | LOC115706751 | (guanine-N(7))- | |
| | | | | | methyltransferase RID2, | |
| | | | | | transcript variant X1 | |
| NC_044371.1 | 1 | 21433547 | 21436754 | gene = | product = 18S rRNA | |
| | | | | LOC115706751 | (guanine-N(7))- | |
| | | | | | methyltransferase RID2, | |
| | | | | | transcript variant X3 | |
| NC_044371.1 | 1 | 21433549 | 21437041 | gene = | product=18S rRNA | |
| | | | | LOC115706751 | (guanine-N(7))- | |
| | | | | | methyltransferase RID2, | |
| | | | | | transcript variant X2 | |
| NC_044371.1 | 1 | 21437550 | 21440586 | gene = | product = general | |
| | | | | LOC115706756 | transcription factor IIF | |
| | | | | | subunit 2 | |
| NC_044371.1 | 1 | 21447348 | 21462402 | gene = | product = beta-taxilin, | M57 |
| | | | | LOC115706737 | transcript variant X1 | |
| NC_044371.1 | 1 | 21447348 | 21462402 | gene = | product = beta-taxilin, | M57 |
| | | | | LOC115706737 | transcript variant X2 | |
| NC_044371.1 | 1 | 21447348 | 21462402 | gene = | product = beta-taxilin, | M57 |
| | | | | LOC115706737 | transcript variant X3 | |
| NC_044371.1 | 1 | 21447348 | 21462402 | gene = | product = beta-taxilin, | M57 |
| | | | | LOC115706737 | transcript variant X4 | |
| NC_044371.1 | 1 | 21447348 | 21462402 | gene = | product = beta-taxilin, | M57 |
| | | | | LOC115706737 | transcript variant X5 | |
| NC_044371.1 | 1 | 21447348 | 21462402 | gene = | product = beta-taxilin, | M57 |
| | | | | LOC115706737 | transcript variant X6 | |
| NC_044371.1 | 1 | 21447348 | 21462380 | gene = | product = beta-taxilin, | M57 |
| | | | | LOC115706737 | transcript variant X8 | |
| NC_044371.1 | 1 | 21474635 | 21477538 | gene = | product = elongation | |
| | | | | LOC115706739 | factor 1-alpha | |
| NC_044371.1 | 1 | 21477812 | 21479214 | gene = | product = uncharacterized | M58, M59 |
| | | | | LOC115706758 | LOC115706758 | |
| NC_044371.1 | 1 | 21483096 | 21486104 | gene = | product = heat shock | |
| | | | | LOC115706731 | protein 83 | |

This principle can be applied by identifying parental markers for any or all of the genes listed in Table 2, including but not limited to markers at the positions of the markers in Table 1. AF and Value Phenotype parents in a given cross can be genotyped for various markers in this or nearby regions of chromosome 1 to identify which loci are polymorphic as to the two parents in the cross. At any locus with an allele pair, if the autoflower parent has one allele and the Value Phenotype parent has the other allele in the pair, the alleles at such locus are then identified as a “Useful Allele Pair.” Progeny of a given cross can be screened for one or more Useful Allele Pairs to confirm individual progeny with desirable recombinations of chromosome 1. Such progeny would carry the autoflower allele of the autoflower parent but with a reduced number of other chromosome 1 alleles of the autoflower parent. For example, each F2 individual showing the AF trait can be scored to determine the number of such markers that correspond to those of the Value Phenotype parent versus the number of such markers that correspond to the AF parent. In this approach, even in the absence of defining which gene from Table 2 causes the AF trait, linkage drag can be reduced by selecting for progeny showing the AF phenotype that also show the fewest AF-parent markers. In a situation in which the specific gene causing the AF trait is known, progeny of any cross can be screened for presence of the specific AF allele and absence of AF-parent alleles at any or all of the other loci in this region of chromosome 1. Thus, it is within the scope of the present invention to use the markers from Table 1 to define a region of chromosome 1 in which to identify markers useful for reducing linkage drag in breeding AF Value Phenotype plants. It is further within the scope of the present invention to address any or all of the genes listed in Table 2 to screen in favor of Value Phenotype parental alleles for these genes, and against AF parent alleles for these genes, with the exception of the AF gene or in the presence of an AF phenotype in the plants thus screened.

In a method of backcrossing, the autoflower trait can be introgressed into a parent having the Value Phenotype (the recurrent parent) by crossing a first plant of the recurrent parent with a second plant having the autoflower trait (the donor parent). The recurrent parent is a plant that does not have the autoflower trait but possesses a Value Phenotype. The progeny resulting from a cross between the recurrent parent and donor parent is referred to as the F1 progeny. One or several plants from the F1 progeny can be backcrossed to the recurrent parent to produce a first-generation backcross progeny (BC1). One or several plants from the BC1 can be backcrossed to the recurrent parent to produce BC2 progeny. This process can be performed for one, two, three, four, five, or more generations. At each generation including the F1, BC1, BC2 and all subsequent generations, the population can be screened for the presence of the autoflower allele using a SNP previously found to be diagnostic of AF. In principle, the progeny resulting from the process of crossing the recurrent parent with the autoflower donor parent are heterozygous for one or more genes responsible for autoflowering. When appropriate, the last backcross generation can be selfed and screened for individuals homozygous for the autoflower allele in order to provide for pure breeding (inbred) progeny with Autoflower Value Phenotype.

In a method of backcrossing, at each generation including the F1, BC1, BC2 and all subsequent generations, the population can be screened with one or more additional background markers throughout the genome that are not known to be associated with the autoflower trait. These selected markers throughout the genome are known to be polymorphic between the recurrent parent and the donor parent. The background markers can be utilized to select against the donor parent alleles throughout the genome in favor of the recurrent parent alleles. The background markers can be utilized to preferentially select progeny at each generation including the F1, BC1, BC2 and all subsequent generations that also exhibit the presence of the desired autoflower allele(s).

Recombinant target markers can be used to identify favorable or unfavorable alleles proximal to the desired target autoflower trait.

In some embodiments, the markers can be defined by their position on chromosome 1, in various ways, for example, in terms of physical position or genetic position. In some embodiments, the markers can be defined by their physical position on chromosome 1, expressed as the number of base pairs from the beginning of the chromosome to the marker (using CS10 as the reference genome). In some embodiments, the markers can be defined by their genetic position on chromosome 1, expressed as the number of centimorgans (a measure of recombination frequency) from the beginning of the chromosome to the marker. In other embodiments, a marker can be defined based upon its location within a given QTL.

### QTL-Based Evidence

Based on the evidence for linkage between autoflower locus and loci involved in agronomic and composition traits, markers were developed to enable the breaking of unfavorable linkage between the autoflower phenotype and other value traits. The use of such markers allows for selection of recombination events between the autoflower locus and other loci involved in other value traits, on chromosome 1, where the autoflower locus is located.

These markers were grouped into marker intervals for simplification purposes. See tables below.

TABLE 3
Marker intervals and number of markers in each region:

| | Beginning Position | Ending Position | |
|---|---|---|---|
| Interval | (BP) | (bp) | Markers |
| Marker Interval 1 (MI1) | 12,331,257 | 14,433,647 | M101, M102 |
| Marker Interval 2 (MI2) | 16,178,336 | 18,018,650 | M103, M104, M105, |
| | | | M106, M107 |
| Marker Interval 3 (MI3) | 19,717,871 | 19,958,734 | M108, M109, M110, |
| | | | M111 |
| Marker Interval 3b | 19,985,933 | 19,992,033 | |
| Marker Interval 4 (MI4) | 19,994,256 | 20,030,132 | M112, M113, M114 |
| Marker Interval 5 (MI5) | 23,557,346 | 39,266,953 | M115, M116, M117, |
| | | | M118 |
| Marker Interval 6 (MI6) | 58,074,007 | 60,618,753 | M119, M120, M121, |
| | | | M122 |
| Marker Interval 7 (MI7) | 80,065,016 | 90,967,989 | M123, M124, M125 |

The markers correlate to the following.

TABLE 4

| | | | Physical | | | | |
|---|---|---|---|---|---|---|---|
| Marker | Marker | | Position | | Ref | Alt | |
| Num | Interval | Chr. | (bp) | SNP | Allele | Allele | Sequence |
| M101 | 1 | 1 | 12331257 | A/G | A | G | ATATTACTTTATATGGTGT |
| | | | | | | | TTTTCTACATTGCTGGTTC |
| | | | | | | | TTTACAATTATTATGGAT |
| | | | | | | | GAGACTAAAATCAAGCTT |
| | | | | | | | TGCGAAAGTGGTTTTGTT |
| | | | | | | | TCATTTCA[A/G]TTTTCAC |
| | | | | | | | TGGGTTGATTTAGATTGTT |
| | | | | | | | ATTGCTAACTTAAGTGCT |
| | | | | | | | GTCTTTGTTTTCTGTTCGG |
| | | | | | | | TGTTCTTTTTCGTACCTAC |
| | | | | | | | CAACTAATGCTCACTTTA |
| | | | | | | | (SEQ ID NO. 119) |
| M102 | 1 | 1 | 14433647 | A/G | A | G | GTCAACATTGGTCTCACC |
| | | | | | | | ATCATCCCCACCATAGCC |
| | | | | | | | AAAAGTAGGAAGGGTGG |
| | | | | | | | TGGTCCCACAAACACTTG |
| | | | | | | | GAGTCCCTCGGGGCTCCT |
| | | | | | | | AAAGAAATTCT[A/G]CTAC |
| | | | | | | | GCCCTCAGCTCGGAGAGC |
| | | | | | | | CTTTTGAACCATCTTAGC |
| | | | | | | | GTAGGTTGTTGTGTCATTT |
| | | | | | | | ATTGTAATGACCAGATCG |
| | | | | | | | TGTCTAGTATTGGCATTC |
| | | | | | | | AAACC (SEQ ID NO. 120) |
| M103 | 2 | 1 | 16178336 | A/G | A | G | TCATTTCTTAGTTACTAAG |
| | | | | | | | AAACTTTTACTTCTAGGA |
| | | | | | | | CGCTACATTAAATCCTAC |
| | | | | | | | ATACTCCTAATTACCCAA |
| | | | | | | | ATACCAATATTATTAACT |
| | | | | | | | TATCACAAT[A/G]TTTCCA |
| | | | | | | | TTAATTCTATTAATTAAGC |
| | | | | | | | ATGTTATGACAATTTTCG |
| | | | | | | | CCCCCGATCGAGTTTTCA |
| | | | | | | | AGATCGCCAAACCTGAAG |
| | | | | | | | ATATTTTTATTTCATATAT |
| | | | | | | | AG (SEQ ID NO. 121) |
| M104 | 2 | 1 | 16447593 | T/A | T | A | ATCTTTAAATAATGAAAA |
| | | | | | | | CTTTTGGAATTGTTCAAGT |
| | | | | | | | AATGCAAATGTGTCAGAA |
| | | | | | | | CGTAACAGAATAAATGTG |
| | | | | | | | CAGTCATTGGTTGAAATG |
| | | | | | | | GAGGAATCA[T/A]TAGAC |
| | | | | | | | AAAGATCTTGAGGAAGCT |
| | | | | | | | CAAGAGCTTAGACATAGA |
| | | | | | | | TGTGAAATTGAAGAAAGA |
| | | | | | | | AATGCTCTCAAAGCTTAT |
| | | | | | | | CGTAAAGCTCAAAGGGAT |
| | | | | | | | CTGGT (SEQ ID NO. 122) |
| M105 | 2 | 1 | 17965679 | G/T | G | T | TTTCTGTAATTACCATCTC |
| | | | | | | | GTGAGAAATAATAACTTG |
| | | | | | | | AAGGGCATGAAATCCATT |
| | | | | | | | AACAAAGTCAAAATATAA |
| | | | | | | | TTTATGAATTTTATTGATT |
| | | | | | | | GAAACTAA[G/T]ATTAAAT |
| | | | | | | | TGAAATTATGCTTTATTA |
| | | | | | | | AGGGCATGAAATCCAACA |
| | | | | | | | AATTGCATGAGCACAAAA |
| | | | | | | | ATAGTGGTTCTCTCACATT |
| | | | | | | | TAAAAATTGAAATTTGAA |
| | | | | | | | AT (SEQ ID NO. 123) |
| M106 | 2 | 1 | 18016243 | C/T | C | T | CTCTTCATTGTAATATAGT |
| | | | | | | | TGGCAAACTCCCAGCCGG |
| | | | | | | | ATCATCCTCCTAGAAGCA |
| | | | | | | | GTTTAGTATCAAAACAAT |
| | | | | | | | CAGCTTCATCCCCTGGGT |
| | | | | | | | TAAATTCCT[C/T]GGGTGC |
| | | | | | | | TGGGGCTCGAAGACCATC |
| | | | | | | | ATCATCAGGTGGTCCAGG |
| | | | | | | | ATCAAGGCCTGGGACTCC |
| | | | | | | | AACTGGACGACCCTCCTT |
| | | | | | | | GAATACTGCATCAAGACC |
| | | | | | | | ATCC (SEQ ID NO. 124) |
| M107 | 2 | 1 | 18018650 | A/G | A | G | TAGAACACTATTCAACTA |
| | | | | | | | AAAACGAAAAAAACGAC |
| | | | | | | | TTCTCACTTGGTGGGGAA |
| | | | | | | | GGAAAGCTGTAAAGGGA |
| | | | | | | | AAACGAAGGGAACAAGA |
| | | | | | | | GTAATTTGATAAG[A/G]G |
| | | | | | | | AGCAATTATTAACCTTCT |
| | | | | | | | CAGAGAAAAGAAGGAAA |
| | | | | | | | GGGTAGAAGAATACAAG |
| | | | | | | | AGACAATAATTTGGGACA |
| | | | | | | | ACATGATTGCATAAGTAG |
| | | | | | | | ATAATTTGGTG (SEQ ID |
| | | | | | | | NO. 125) |
| M108 | 3 | 1 | 19717871 | G/A | G | A | GTTGTTGCATTTTAAGCCT |
| | | | | | | | TTTGAAATATTTGTCTAG |
| | | | | | | | AGTCTCTGATCTCATTTTC |
| | | | | | | | TATAGTAAACTAACTGCT |
| | | | | | | | TTATTTGTTTCTTTGTTGT |
| | | | | | | | GTATGTA[G/A]AGCAAGT |
| | | | | | | | GAAGGAGGGGTCCCACAT |
| | | | | | | | GTACTATATAAGGGATTA |
| | | | | | | | TGGGGGGAACCCTTTTCA |
| | | | | | | | CTTTTTACTTTTTACTTCT |
| | | | | | | | CTCACTATAAATTGAAAT |
| | | | | | | | TT (SEQ ID NO. 126) |
| M109 | 3 | 1 | 19812701 | T/C | T | C | TGCTTACAAAAATAGCAC |
| | | | | | | | TGTCTTCTATAACTCCAAC |
| | | | | | | | AAAGTAAAGTTCCAAAAG |
| | | | | | | | TAGCTTCAGAGTACTGCG |
| | | | | | | | CTTCTTCATAGCCTTTTGA |
| | | | | | | | TTCCTATC[T/C]GTATCTG |
| | | | | | | | AGTCATCCCCTGATTTTCC |
| | | | | | | | AGGAAAGAAGACTTTCAA |
| | | | | | | | AAGCCCTTGAACAAGGCT |
| | | | | | | | CGGGGAGAAGTCTTTGTA |
| | | | | | | | TCTTTGATGAAGCAAAGA |
| | | | | | | | GC (SEQ ID NO. 127) |
| M110 | 3 | 1 | 19950755 | T/C | T | C | TCTCAACAAACAGGTTCG |
| | | | | | | | GAACAGTAAAAAATTGAT |
| | | | | | | | GGATTCATTCTATTTATAA |
| | | | | | | | AGCAAAAATAATAACAA |
| | | | | | | | GTGTTATAACGAGATTTC |
| | | | | | | | TAAGATCAAT[T/C]TTAAT |
| | | | | | | | AGTAAAATGAGAAGTTAA |
| | | | | | | | ATGTAAAAGGATGAACTA |
| | | | | | | | AATACTCGCATATGTCAA |
| | | | | | | | AACAAAAACACCAAAAA |
| | | | | | | | TAACTAAGTATAAAATTA |
| | | | | | | | GTTCTA (SEQ ID NO. 128) |
| M111 | 3 | 1 | 19958734 | C/G | C | G | TTTCTAGTCTTTGGTTCTT |
| | | | | | | | CCAGGTGGAGATTATTTA |
| | | | | | | | CTTTGTCTTCATAGTGGA |
| | | | | | | | AAATTTGGGATTTTGGAC |
| | | | | | | | TCACAGAGTTAGCTCACT |
| | | | | | | | TCTCCTTTC[C/G]AGGGCG |
| | | | | | | | ATGTCGCGAGGTACCGAG |
| | | | | | | | ATCTCGCGGTGTTAGTAG |
| | | | | | | | CAGAGGGTGCCTCAGTGA |
| | | | | | | | TGATGGGACCTCCTTCCTT |
| | | | | | | | TGTATCATACATTTACCTT |
| | | | | | | | CG (SEQ ID NO. 129) |
| M112 | 4 | 1 | 19994256 | G/T | G | T | TGAGGAATTGGCCACCCC |
| | | | | | | | AAGGCTTTTTCTAGTTGCC |
| | | | | | | | TAGCCCGCGCAGTAATTA |
| | | | | | | | AGATAAGCCTTCTTGGAG |
| | | | | | | | TCTCCGAGGTAATCAAAA |
| | | | | | | | TTGCCTGCA[G/T]TGTTTG |
| | | | | | | | CCTTCTAGAATTCATAAA |
| | | | | | | | AGACCTACAGGGCGGTAG |
| | | | | | | | TTTCCAAATTCTCGACCTC |
| | | | | | | | CTTCGAGAGCTCTTCTTCC |
| | | | | | | | CTCGTCTGCCTGGCCTTA |
| | | | | | | | AC (SEQ ID NO. 130) |
| M113 | 4 | 1 | 20011591 | A/T | A | T | TCTGCTCTGTGATGGGAT |
| | | | | | | | CCTGTACCTCCTCATTAGT |
| | | | | | | | ACACTTTCCATCATTCGTT |
| | | | | | | | GTCATCTCCTCAGATTTCA |
| | | | | | | | CCTGTTTCATTCAAGCAA |
| | | | | | | | ATATTAG[A/T]ACATAACA |
| | | | | | | | AAAACACTATACTTCCAA |
| | | | | | | | GCTTTATAATGTGACCAT |
| | | | | | | | CATGGTAAACCAGCAAAC |
| | | | | | | | GCACCATCATGTTATTTA |
| | | | | | | | CAAATGAAGCTAACCATA |
| | | | | | | | TT (SEQ ID NO. 131) |
| M114 | 4 | 1 | 20030132 | C/T | C | T | ATGTAGTCCTGGTCATCC |
| | | | | | | | ATCCATCTCAGGAATTTG |
| | | | | | | | CGTCTCTGTTGGGTGAAT |
| | | | | | | | GCATCACAACCTACAACA |
| | | | | | | | CTTGGTTCTTCAGTTTGGA |
| | | | | | | | CTTGGCTTA[C/T]AGCCTC |
| | | | | | | | TCTAAGTATGGCCAACTA |
| | | | | | | | AAAGTCTTCTCCTAAGTC |
| | | | | | | | CACATGAGGCTCACCAAT |
| | | | | | | | TGTTGGTTCGCCAATTCCT |
| | | | | | | | GGCCCTATTTCCCCAGCA |
| | | | | | | | TAA (SEQ ID NO. 132) |
| M115 | 5 | 1 | 23557346 | T/C | T | C | CTCACCACACTTAGCATA |
| | | | | | | | CATGTCAATGAGCGAGGT |
| | | | | | | | CGTGAGATGACAGTTTAA |
| | | | | | | | CTTAAAACCCTGCCTTCC |
| | | | | | | | AATGTAGACATGAATCCA |
| | | | | | | | CCTGCCAGGA[T/C]CAATA |
| | | | | | | | GCTCCTAACTGGGAACAA |
| | | | | | | | GCTGATAGTGTACTGACC |
| | | | | | | | AAAGTGATTTGATCAGGT |
| | | | | | | | TTTACACTCTTGCTAAGTT |
| | | | | | | | GCAATTGATGGAAAACAG |
| | | | | | | | CCAA (SEQ ID NO. 133) |
| M116 | 5 | 1 | 23715246 | C/G | C | G | GATTCTTCTTTGTACCATG |
| | | | | | | | TTTTTGATTTTGGAAGTTG |
| | | | | | | | ATGTTGTCTCTTCAAGTCT |
| | | | | | | | AGGACAAAGAAGAAATG |
| | | | | | | | AGATGTTTAAGAACTAAA |
| | | | | | | | ATCAAAAC[C/G]AATCTTA |
| | | | | | | | ATAGTGATGTTATCTAGT |
| | | | | | | | TAGCTTACCACAAATGTC |
| | | | | | | | ACCCTTGACTCTTCCCAG |
| | | | | | | | GCTTTCAGAGCTAAATGG |
| | | | | | | | CAGTTCCAGCACAGAAAT |
| | | | | | | | TGG (SEQ ID NO. 134) |
| M117 | 5 | 1 | 24577079 | T/A | T | A | ACTCGAAAATCCAAGTGT |
| | | | | | | | GGAATAATGGCTGGTCTT |
| | | | | | | | GTGGCGATGGTGTTATTA |
| | | | | | | | TTGATGCTAGTGCAATCA |
| | | | | | | | ATCCTCATGTGGTGACGG |
| | | | | | | | ATTTACCTAT[T/A]CATGC |
| | | | | | | | ATTTTTAACAAAGAATGG |
| | | | | | | | AGTAGAGTGGAACACGAC |
| | | | | | | | TGAAGTGAAATGGGTGTT |
| | | | | | | | CAGGCCTTCGATTGCCGA |
| | | | | | | | GGTAATCTTGAATTGTAG |
| | | | | | | | GACTG (SEQ ID NO. 135) |
| M118 | 5 | 1 | 39266953 | G/A | G | A | TCAGTTCTTTTATTTTTAA |
| | | | | | | | TTTTTTGCGTGACACAGTC |
| | | | | | | | AGTTCTTCCTTGTTGATGT |
| | | | | | | | TCGATTGAATCTCTCTCAT |
| | | | | | | | ATTGACTACTTGTAATTTG |
| | | | | | | | TTGTT[G/A]CAGCGGGAAT |
| | | | | | | | TCCGAATGTCAGGTGGTT |
| | | | | | | | TGGAGTTGAAGGAGAGTA |
| | | | | | | | TAATGTTTTGGTGATGGA |
| | | | | | | | TTTGCTGGGTCCTAGTCTT |
| | | | | | | | GAAGATCTCTTTAATTT |
| | | | | | | | (SEQ ID NO. 136) |
| M119 | 6 | 1 | 58074007 | C/A | C | A | CAAGTCATTGATATCATA |
| | | | | | | | CCTCCAGTTAGAGATAAG |
| | | | | | | | ATGAAGGTGCACTAGTAT |
| | | | | | | | AACGCAGTGGAGCATCAT |
| | | | | | | | ATGGATGTGCCCAGCAAA |
| | | | | | | | CATTATCAAG[C/A]ACAG |
| | | | | | | | GAGACATTTTCAAGCCAA |
| | | | | | | | TGATCTGTACATCTGGAT |
| | | | | | | | TTGAGTGACCATCGAGAA |
| | | | | | | | AAATATTTGTAATCTACA |
| | | | | | | | TAGAAAAGAAAACATAA |
| | | | | | | | CCCAACC (SEQ ID NO. 137) |
| M120 | 6 | 1 | 59149195 | C/G | C | G | TACAATGATAATAACAAA |
| | | | | | | | ATGAAACAACAAAACCAT |
| | | | | | | | GATAATCACAAGAATTTG |
| | | | | | | | ATGGGAGTGAAAGTGATC |
| | | | | | | | ATTCCGAACCATAAACGC |
| | | | | | | | TAGCTAATAC[C/G]AATCA |
| | | | | | | | ATACACTCCAAAATAATG |
| | | | | | | | AATTTTCTGTTTGAGGAT |
| | | | | | | | AACAATCTTAGTTTATTTA |
| | | | | | | | ATCATTCAAAAGGATTGA |
| | | | | | | | ATAATCTGATGAAGAACA |
| | | | | | | | TGAT (SEQ ID NO. 138) |
| M121 | 6 | 1 | 59926686 | C/T | C | T | ACTAGTTACCAAATACAA |
| | | | | | | | TCAATTGAAAAAACAGAA |
| | | | | | | | ACAAATATATAAATCACC |
| | | | | | | | TAAAATAATAAAACATAA |
| | | | | | | | ATTAAAATACAAAAATCA |
| | | | | | | | ATTACAAAAT[C/T]ACCTG |
| | | | | | | | AAATAAATTTATAAATTA |
| | | | | | | | TTTTTGTAAATGCTAGTTA |
| | | | | | | | CAGAATTTTTTTAGCTAGT |
| | | | | | | | TTATTACTTCACTCTGCAT |
| | | | | | | | TTTGTGCAATATCATCGA |
| | | | | | | | AT (SEQ ID NO. 139) |
| M122 | 6 | 1 | 60618753 | G/T | G | T | TTGTTTTATCGACACTAG |
| | | | | | | | AGAGGAGGTTCTTAGAGA |
| | | | | | | | ATGATAAATGATCCATTC |
| | | | | | | | ATCATGCTGACTTATGTT |
| | | | | | | | ATGATGACTTCATTGTTG |
| | | | | | | | CGCAACCATT[G/T]CATGA |
| | | | | | | | ACAAGTTATGGGAATTGA |
| | | | | | | | TCGTAACCTTGAGGAAGA |
| | | | | | | | CGATGCTGAATACATTAG |
| | | | | | | | AAACGATATTAATGAGGG |
| | | | | | | | AATATGGGTAAATTGTGA |
| | | | | | | | TTCAG (SEQ ID NO. 140) |
| M123 | 7 | 1 | 80065016 | C/T | C | T | CCAGGTTCAAGTCCCCAT |
| | | | | | | | GATTGTGCATGAATACGA |
| | | | | | | | AGATCGGTAATGAACATA |
| | | | | | | | ATCAGTGGATTAATATGT |
| | | | | | | | TACTTTTTCATGGTTATAT |
| | | | | | | | ATCATGGAG[C/T]TAGTCA |
| | | | | | | | TTCAATTTCAAAATAAGA |
| | | | | | | | AAATGATAATAACTATGG |
| | | | | | | | GTTGAAATTGGGAAAATT |
| | | | | | | | GTTGCTAGAGGTGGGTGA |
| | | | | | | | ACCAACTTCATTAGGGGT |
| | | | | | | | TTGA (SEQ ID NO. 141) |
| M124 | 7 | 1 | 85078747 | G/C | G | C | AACCGCCCGCCGTCACGC |
| | | | | | | | ATAGCCCGTCTCCAACCA |
| | | | | | | | CCTGCTGCTTATCTTCATC |
| | | | | | | | TCTTTAAGTTCTATTTGTA |
| | | | | | | | AGTTCTTTTTCCTCTTCTT |
| | | | | | | | AATTTTT[G/C]GTAACAAA |
| | | | | | | | TATTTAGTTTTGGCTGTAA |
| | | | | | | | CGGTAACAAATATTTGGT |
| | | | | | | | GTTGGCTGCTATTTTAAC |
| | | | | | | | ATTTTTGTATAGATTAAG |
| | | | | | | | AATGATTTCAATCTCTGCT |
| | | | | | | | (SEQ ID NO. 142) |
| M125 | 7 | 1 | 90967989 | A/G | A | G | GTATGTGTAGAGGGAGTA |
| | | | | | | | CAAGCAGTGATGTTAGTG |
| | | | | | | | ATGAGAGCAGTTGTAGCA |
| | | | | | | | GCTTTAGTAGCAGTGTAA |
| | | | | | | | ACAAACCTCACAAAGCGA |
| | | | | | | | ATGACTTCAA[A/G]TGGG |
| | | | | | | | AAGCTATCCAAGCTGTCC |
| | | | | | | | GAGAAAAAGAGGGGATG |
| | | | | | | | CTCGGTTTGACACATTTTA |
| | | | | | | | GACTGCTAAAGAGGTTGG |
| | | | | | | | GTTGTGGGGATATTGGAA |
| | | | | | | | GTGTTT (SEQ ID NO. 143) |

As used in reference to Table 3, and treating Marker Interval 3b as being an interval of interest correlating with the autoflower phenotype, “upstream” of the interval of interest can be defined by: Any individual marker or group of markers within MI2 (alone or together with one or more markers from within MI1), can be used to select for recombination between the interval of interest and QTLs located within QTI1 and beyond (all the way to the end of the short arm of Chromosome 1), and therefore to break unfavorable associations between the autoflower phenotype associated with the interval of interest and all value traits explained by those QTLs. (QTI=QTL Interval)

Any individual marker or group of markers within MI3 (alone or together with one or more markers from within MI1, MI2, or MI1 and MI2), can be used to select for recombination between the interval of interest and QTLs located within QTI2 or QTI1 and beyond (all the way to the end of the short arm of Chromosome 1), and therefore to break unfavorable associations between the autoflower phenotype associated with the interval of interest and all value traits explained by those QTLs.

As used herein “Downstream” of the interval of interest can be defined by: Any individual marker or group of markers within MI4 (alone or together with one or more markers from within MI5, MI6, MI7, MI5 and MI6, MI5 and MI7, MI6 and MI7, or MI5 and MI6 and MI7), can be used to select for recombination between the interval of interest and QTLs located within QTI3, QTI4 or QTI5 and beyond (all the way to the end of the long arm of Chromosome 1), and therefore to break unfavorable associations between the autoflower phenotype associated with the interval of interest and all value traits explained by those QTLs.

Any individual marker or group of markers within MI5 (alone or together with one or more markers from within MI6, MI7, or MI6 and MI7), can be used to select for recombination between the interval of interest and QTLs located within QTI4 or QTI5 and beyond (all the way to the end of the long arm of Chromosome 1), and therefore to break unfavorable associations between the autoflower phenotype associated with the interval of interest and all value traits explained by those QTLs.

Any individual marker or group of markers within MI6 (alone or together with one or more markers from within MI7), can be used to select for recombination between the interval of interest and QTLs located within QTI5 and beyond (all the way to the end of the long arm of Chromosome 1), and therefore to break unfavorable associations between the autoflower phenotype associated with the interval of interest and all value traits explained by those QTLs.

Where another interval of interest correlates strongly with the autoflower phenotype, and is in a locus outsideMI3b, the use of the other groups of markers as discussed above would be adjusted accordingly.

As used herein “upstream” and “downstream” of the autoflower locus can be defined by: Any combination of one of the above “upstream” and one of the above “downstream” processes can be used to select for recombinations simultaneously on both sides of the interval of interest, and therefore to break unfavorable associations between the autoflower phenotype associated with the interval of interest and all value traits explained by the respective QTLs.

### Gene-Based Evidence

Based on the evidence for linkage between autoflower locus and loci involved in agronomic and composition traits, markers were developed to enable the breaking of unfavorable linkage between the autoflower phenotype and other value traits. The use of such markers allows for selection of recombination events between the autoflower locus and other loci involved in other value traits, on chromosome 1, where the autoflower locus is located.

These markers were grouped into marker intervals for simplification purposes as in Table 3.

Alleles causing an autoflower phenotype can be in one or more marker intervals or regions of chromosome 1. For example, treating MI3b as an interval of interest associated with the autoflower phenotype, as used herein, “upstream” of the interval of interest can be defined by: any individual marker or group of markers within MI2 (alone or together with one or more markers from within MI1), can be used to select for recombination between the interval of interest and genes located within GI1 and beyond (all the way to the end of the short arm of Chromosome 1), and therefore to break unfavorable associations between the autoflower phenotype associated with the interval of interest and all value traits explained by those genes. (GI=Gene Interval)

Treating MI3b as an interval of interest, any individual marker or group of markers within MI3 (alone or together with one or more markers from within MIL MI2, or MI1 and MI2), can be used to select for recombination between the interval of interest and genes located within GI2 or GI1 and beyond (all the way to the end of the short arm of Chromosome 1), and therefore to break unfavorable associations between the autoflower phenotype associated with the interval of interest and all value traits explained by those genes.

Likewise, treating MI3b as an interval of interest, some individuals marker or group of markers within MI3 (alone or together with one or more markers from within MI1, MI2, or MI1 and MI2), can be used to select for recombination between the interval of interest and genes located within GI3, and therefore to break unfavorable associations between the autoflower phenotype associated with the interval of interest and all value traits explained by those genes.

Treating MI3b as an interval of interest, as used herein “Downstream” of the interval of interest can be defined by: some individual marker or group of markers within MI4 (alone or together with one or more markers from within MI5, MI6, MI7, MI5 and MI6, MI5 and MI7, MI6 and MI7, or MI5 and MI6 and MI7), can be used to select for recombination between the interval of interest and genes located within GI4, and therefore to break unfavorable associations between the autoflower phenotype associated with the interval of interest and all value traits explained by those genes.

Treating MI3b as an interval of interest, any individual marker or group of markers within MI4 (alone or together with one or more markers from within MI5, MI6, MI7, MI5 and MI6, MI5 and MI7, MI6 and MI7, or MI5 and MI6 and MI7), can be used to select for recombination between the interval of interest and genes located within GI5, GI6 or GI7 and beyond (all the way to the end of the long arm of Chromosome 1), and therefore to break unfavorable associations between the autoflower phenotype associated with the interval of interest and all value traits explained by those genes.

Treating MI3b as an interval of interest, any individual marker or group of markers within MI5 (alone or together with one or more markers from within MI6, MI7, or MI6 and MI7), can be used to select for recombination between the interval of interest and genes located within G16 or GI7 and beyond (all the way to the end of the long arm of Chromosome 1), and therefore to break unfavorable associations between the autoflower phenotype associated with the interval of interest and all value traits explained by those genes.

Treating MI3b as an interval of interest, any individual marker or group of markers within MI6 (alone or together with one or more markers from within MI7), can be used to select for recombination between the interval of interest and genes located within GI7 and beyond (all the way to the end of the long arm of Chromosome 1), and therefore to break unfavorable associations between the autoflower phenotype associated with the interval of interest and all value traits explained by those genes.

As used herein “upstream” and “downstream” of the interval of interest can be defined by: Any combination of one of the above “upstream” and one of the above “downstream” processes can be used to select for recombinations simultaneously on both sides of the interval of interest, and therefore to break unfavorable associations between the autoflower phenotype and all value traits explained by the respective genes. Where one or more other intervals of interest are strongly associated with an autoflower phenotype, the same principles as discussed herein can apply to flanking intervals to minimize linkage drag in breeding steps to introgress an autoflower trait into a Value Phenotype.

### Breeding Methods

The methods provided herein can be used for detecting the presence of the autoflower trait markers in Cannabis plant or germplasm, and can therefore be used in methods involving marker-assisted breeding and selection of Cannabis plants having the autoflower phenotype.

Thus, methods for identifying, selecting and/or producing a Cannabis plant or germplasm with the autoflower trait can comprise detecting the presence of a genetic marker associated with the autoflower trait. The marker can be detected in any sample taken from a Cannabis plant or germplasm, including, but not limited to, the whole plant or germplasm, a portion of said plant or germplasm (e.g., a cell, leaf, seed, etc, from said plant or germplasm) or a nucleotide sequence from said plant or germplasm.

Breeding methods can include recurrent, bulk or mass selection, pedigree breeding, open pollination breeding, marker assisted selection/breeding, double haploids development and selection breeding. Double haploids are produced by the doubling of a set of chromosomes (1 N) from a heterozygous plant to produce a completely homozygous individual.

The invention relates to molecular markers and marker-assisted breeding of autoflower Cannabis plants. Specifically, in the context of breeding to develop Autoflower Value Phenotype varieties, a molecular marker correlating strongly with the autoflower trait can permit very early testing of progeny of a cross to identify those progeny that possess one or more autoflower alleles and discard those individuals that do not. This permits shifting the allele frequency of any plants remaining in the breeding pool, after such screening, to eliminate any plants that do not have at least one autoflower allele. In some embodiments of the invention, the analysis is capable of distinguishing between individuals that are homozygous for the autoflower allele versus those that are heterozygous. In such situations it can be advantageous to discard any heterozygous individuals.

Additional breeding methods that, in some embodiments, can be combined with marker-assisted breeding are known to those of ordinary skill in the art and include, e.g., methods discussed in Chahal and Gosal (Principles and procedures of plant breeding: biotechnological and conventional approaches, CRC Press, 2002, ISBN 084931321X, 9780849313219); Taji et al. (In vitro plant breeding, Routledge, 2002, ISBN 156022908X, 9781560229087); Richards (Plant breeding systems, Taylor & Francis US, 1997, ISBN 0412574500, 9780412574504); Hayes (Methods of Plant Breeding, Publisher: READ BOOKS, 2007, ISBN1406737062, 9781406737066); each of which is incorporated by reference in its entirety. The Cannabis genome has been sequenced (Bakel et al., The draft genome and transcriptome of Cannabis sativa, Genome Biology, 12(10):R102, 2011). Molecular makers for Cannabis plants are described in Datwyler et al. (Genetic variation in hemp and marijuana (Cannabis sativa L.) according to amplified fragment length polymorphisms, J Forensic Sci. 2006 March; 51(2):371-5.); Pinarkara et al., (RAPD analysis of seized marijuana (Cannabis sativa L.) in Turkey, Electronic Journal of Biotechnology, 12(1), 2009), Hakki et al., (Inter simple sequence repeats separate efficiently hemp from marijuana (Cannabis sativa L.), Electronic Journal of Biotechnology, 10(4), 2007); Gilmore et al. (Isolation of microsatellite markers in Cannabis sativa L. (marijuana), Molecular Ecology Notes, 3(1): 105-107, March 2003); Pacifico et al., (Genetics and marker-assisted selection of chemotype in Cannabis sativa L.), Molecular Breeding (2006) 17:257-268); and Mendoza et al., (Genetic individualization of Cannabis sativa by a short tandem repeat multiplex system, Anal Bioanal Chem (2009) 393:719-726); each of which is herein incorporated by reference in its entirety.

Additional breeding methods that can be used in certain embodiments of the invention, can be found, for example in, U.S. patent Ser. No. 10/441,617B2.

The following examples are included to demonstrate various embodiments of the invention and are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### EXAMPLES

### Example 1

### QTL Detection/Mapping

A quantitative trait locus (QTL) analysis of an auto-flowering (AF) trait was conducted using an F2 pedigree with 192 progeny samples. A single categorical phenotype was measured on the progeny. The phenotype shows a recessive segregation pattern, expressed in approximately 25% of the samples. QTL analysis identified a single locus in perfect correlation with the trait consistent with the recessive model.

### Example 2

### Sequencing

Parents were deep sequenced and progeny of Example 1 were skim sequenced. Genotypes were imputed and haplotype blocks defined. These blocks were tested for association with the autoflower trait.

Sequencing depth varied as follows: 173 samples at 2× coverage, 20 samples at 8× coverage, and a parental line at 30× coverage. The sequencing data for 192 progeny samples passed required QC standards and were used in the QTL analysis. FIG. 1 shows a schematic view of the pedigree including the sequencing depth (note that only one parental line, Banana OG, was sequenced in the analysis).

### Example 3

### Analysis Pipeline/Haplotype Inference

CS10 assembly from NCBI, version: GCA_900626175.2 (www<dot>ncbi nlm nih<dot>gov/assembly/GCF_900626175.2) was used as a reference genome. Chrom-X was changed to Chrom-10 due to technical reasons but no other change to the reference was made.

All samples from Example 2 were mapped to the reference genome followed by a Variant-Calling pipeline using GATK and in-house tools to process the Skim-Seq data optimally. After variant filtration, a total of 45573 SNPs were selected for the next stage. The Variant-Calling procedure was followed by a haplotype-inference algorithm that infers the 2 haplotypes in the F1 generation (A and B, see the diagram below). The segregating genotypes in the progeny were inferred for each sample at each location along the genome. The 3 possible genotypes are designated as follows: AA, AB and BB.

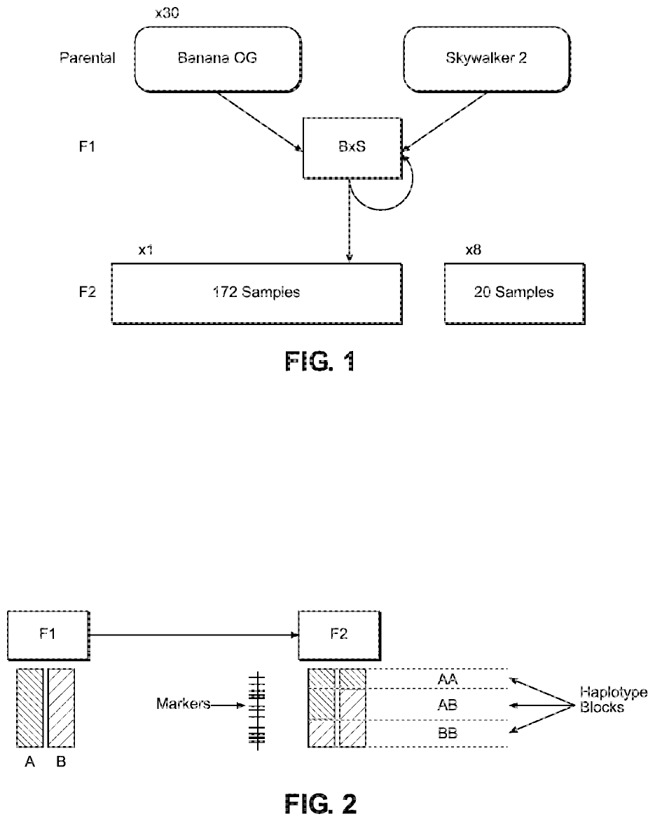

The basic genotyping unit is the haplotype-block (HB), defined as a segment between consecutive recombination events in any of the progeny samples. Within haplotype blocks, there are no recombination events, and all markers (SNPs) could be used to measure sample genotypes. FIG. 2 is a schematic view of haplotype-blocks.

### Example 4

### QTL Analysis

A QTL scan was performed by regressing the phenotype on the genotype at each haplotype-block from FIG. 2. A significant QTL was declared if a model including the genotype was substantially better than a model without the genotype using a likelihood-ratio test. A threshold of FDR<0.01 was used to declare significant results (FDR=false discovery rate).

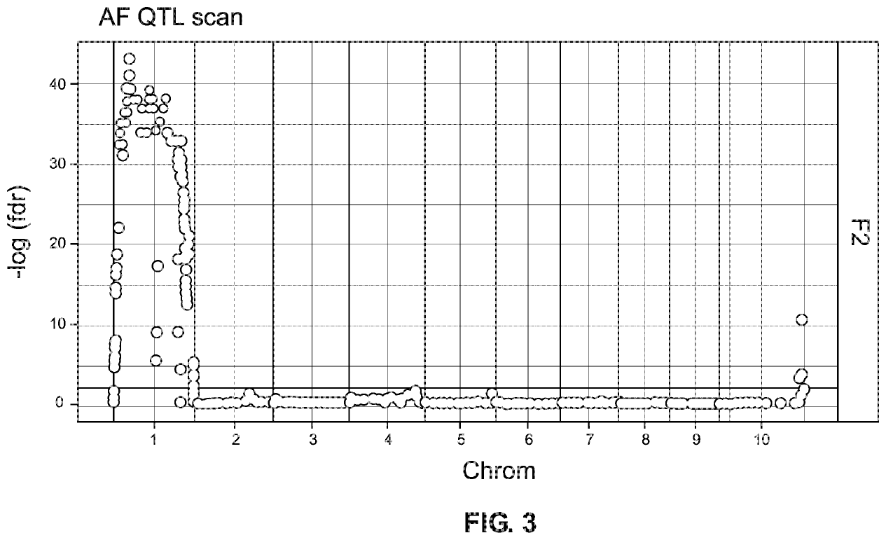

Assuming a categorical (Yes/No) phenotype, a genome-wide scan using logistic regression was implemented. The result is presented in the figure and tables below. The figure shows the FDR values on a log scale for each chromosome on each of the haplotype blocks. The horizontal line indicates a significance threshold (FDR of 0.01). FIG. 3 shows a single QTL peak on Chromosome 1 that is highly significant, along with a minor peak on Chromosome 10.

### Example 5

### Confidence Interval

The table below shows the Confidence Interval (CI) around the peak in Example 4. This interval can be the suggested region for generating markers for the QTL.

TABLE 5

| markerKey | Chrom | CLlow | CLHigh |
|---|---|---|---|
| MK_48 | 1 | 19118486 | 21479285 |

### Example 6

### Effect Size

Below is a summary count of the phenotype values per genotype at the peak on Chromosome 1 in Example 4. Note that there is a perfect match between the phenotype and genotype at this location. The peak on Chromosome 10 is tagged as a false positive since the phenotype/genotype correlation on Chromosome 1 is perfect.

TABLE 6

| | Autoflower | Photoperiod |
|---|---|---|
| Genotype Class | Phenotype Count | Phenotype Count |
| AA | 0 | 60 |
| AB | 0 | 86 |
| BB | 46 | 0 |

### Example 7

### SNP Markers

SNP set was generated to be used as markers for the QTL locus. This SNP set was generated under the assumption that the phenotype is recessive and the causative haplotype is found in a homozygous state in the relevant progeny samples (Phenotype=1). The marker set is provided in Table 1.

### Example 8

### Appendix/SNP Markers File

SNP markers for the segregating allele (i.e., the BB genotype) at the QTL locus were selected based on the following criteria:
At least 100 bp flanking region with no other variant
GC content between 30-70%
Scored well within the haplotype inference algorithm

The data contain the following attributes for each SNP:
Chrom/Pos: Coordinates relative to CS10
Ref/Alt: The reference and alternative alleles relative to CS10
Marker_Allele: the allele linked with the B haplotype
Flanking sequences around the SNP allele
GC content of the flanking sequences

### Example 9

### Haplotype Blocks File

The haplotype-blocks and the sample genotype within each block are provided.

The file contains the location of each haplotype block detected in the analysis together with the assigned genotype of each sample. The genotypes were coded as characters with the following schema:
AA: Homozygous for A allele
BB: Homozygous for B allele
AB: Heterozygous

Note that the A and B alleles are arbitrary and bear no relation to the reference/alternative alleles found in the variant-calling analysis.

### Example 10

### Phenotypic Correlation Between Autoflower and Agronomic or Composition (Value Trait) Performance

Varieties extracted for commercial production were evaluated for different traits including, total cannabinoid concentration, total THC concentration, total terpene concentration (as mg/g of dry matter) and oil yield as % of fresh frozen biomass. Autoflower varieties showed significantly lower cannabinoid, THC, and terpene concentrations, as well as oil yield than the daylength sensitive varieties.

Sample descriptives for total concentration of cannabinoids. THC, terpenes, and oil yield percent.

TABLE 7

| | Cannabinoids Total | | THC Total | | Terpene Total | | Oil Yield | |
|---|---|---|---|---|---|---|---|---|
| | Concentration | | Concentration | | Concentration | | Percent | |
| | (mg/g) | | (mg/g) | | (mg/g) | | % | |
| Class | AF | PP | AF | PP | AF | PP | AF | PP |
| # | 214 | 341 | 214 | 341 | 216 | 154 | 33 | 155 |
| Materials | | | | | | | | |
| Mean | 134 | 207.5 | 121.7 | 183.1 | 3 | 5.4 | 4 | 5.9 |
| Std. | 31.8 | 35.5 | 30.5 | 31.8 | 1.4 | 2.5 | 0.5 | 0.9 |
| Deviation | | | | | | | | |
| P value | | <0.001 | | <0.001 | | <0.001 | | <0.001 |

These results clearly show the relationship between auto-flowering/daylength sensitivity and economically important traits in Cannabis sativa. The auto-flowering characteristic is always/generally associated with lower values of these economically important traits than daylength sensitivity. Because of the genetic structure of these two groups of materials—being selfed progenies of auto-flowering×daylength sensitive segregating crosses—this observation is strong evidence for the existence of negative genetic linkage between the autoflower allele at the auto-flower locus and agronomically and economically desirable traits. Breaking such negative linkage will require specific processes, including the use of specific markers outside of yet closely flanking the autoflower locus.

### Example 11

### Breeding for Improved Autoflower Materials

A number of crosses are made between autoflower lines and PP materials (clones) with the objective of developing autoflower lines with agronomic and composition (value trait or traits) performance similar to that of the PP parent. Large (several hundred) F2 populations are developed and screened for the presence of the autoflower allele using a SNP previously found to be diagnostic of AF. Plants homozygous for the autoflower allele are selected. The selected plants are phenotyped for flowering behavior to confirm their being AF. They are also phenotyped for composition traits, based on which a further selection step is carried out. F2 plants with positive results as to all selection criteria are self-fertilized to generate F3 seed. F3 families are phenotyped for agronomic and composition traits, and selected on the basis of their performance. One or more plants from each selected family are selfed to generate the following generation. This process is followed for a number of generations, up to the F7 generation in a number of cases. All materials from F3 and beyond always show the autoflower phenotype. All, however, also show performance levels significantly lower than day-length sensitive materials for one or more agronomic or composition traits (value traits).

Without wishing to be bound by a particular theory, the difficulty in recovering an agronomically- or compositionally acceptable C. sativa plant with autoflower is most likely the result of linkage drag of undesirable traits from the autoflower sources.

### Example 12

### Marker Assisted Backcrossing

In a method of backcrossing, the autoflower trait is introgressed into a parent having the Value Phenotype (the recurrent parent) by crossing a first plant of the recurrent parent with a second plant having the autoflower trait (the donor parent). The recurrent parent is a plant that does not have the autoflower trait but possesses a Value Phenotype. The progeny resulting from a cross between the recurrent parent and donor parent is referred to as the F1 progeny. One or several plants from the F1 progeny are backcrossed to the recurrent parent to produce a first-generation backcross progeny (BC1). One or several plants from the BC1 are backcrossed to the recurrent parent to produce BC2 progeny. At each generation including the F1, BC1, BC2 and all subsequent generations, the population is screened for the presence of the autoflower allele using a SNP previously found to be diagnostic of AF. The progeny resulting from the process of crossing the recurrent parent with the autoflower donor parent are heterozygous for one or more genes responsible for autoflowering. The last backcross generation is selfed and screened for individuals homozygous for the autoflower allele in order to provide for pure breeding (inbred) progeny with Autoflower Value Phenotype.

### Example 13

### Background Markers

In a method of backcrossing, at each generation including the F1, BC1, BC2 and all subsequent generations, the population is screened with additional background markers throughout the genome that are not known to be associated with the autoflower trait. These selected markers throughout the genome are known to be polymorphic between the recurrent parent and the donor parent. The background markers are utilized to select against the donor parent alleles throughout the genome in favor of the recurrent parent alleles. The background markers are utilized to preferentially select progeny at each generation including the F1, BC1, BC2 and all subsequent generation that also exhibit the presence of the desired autoflower allele(s).

### Example 14

### Association Mapping of Autoflower and Agronomic and Composition Traits (Value Traits)

A set of 267 Cannabis sativa materials, including heterozygous clones and inbred families (F3's and F4's) were selected to form a diverse association mapping (AM) panel. The panel consisted of materials with a wide range of flowering behavior, terpenes, maturity and other agronomic traits.

A set of 267 Cannabis sativa materials, including heterozygous clones and inbred families (F3's and F4's) were selected to form a diverse association mapping (AM) panel. The panel consisted of materials with a wide range of flowering behavior, terpenes, maturity and other agronomic traits.

These materials were phenotyped in 2020 for a number of traits including daylength sensitivity (AF or photo), days to maturity, CBD, THC and a set of terpene profiles.

All materials were genotyped with 600 SNPs and used for the GWAS analysis.

Data analysis: Association mapping based on mixed linear model (MLM) with population structure as a covariate was conducted using TASSEL, a JAVA based open-source software for linkage and association analysis (Bradbury et al., 2007).

Results: The autoflower locus was mapped to chromosome 1 at position 19,988,827 bp (as positions are established in the cs10 reference genome). Significant associations for different terpene profiles and maturity were identified on chromosome 1 as well as other chromosomes.

Significant marker trait associations were used to assign co-segregating or adjacent significant markers into QTL intervals. Markers with the most significant p-values were extracted as representative markers for each marker trait association. Some of the loci were detected for multiple traits, so all those were combined under one QTL interval. The most significant QTLs were positioned based on physical position against the Cs10 Genome Assembly (GCA_900626175.2).

TABLE 8
QTL regions significantly associated with terpene profiles
and days to maturity (p.MLM <0.001), and linked to the
autoflower locus in an interval of interest, on chromosome 1.

| QTL | Beginning | End | | Num |
|---|---|---|---|---|
| Intervals | Position (bp) | Position | Trait | SNPs |
| QTLl1 | 14,443,748 | 15,023,503 | Terpene Profile | 2 |
| QTLl2 | 18,014,544 | 19,290,938 | Terpene Profile, | 3 |
| | | | Days to Maturity | |
| Exemplary | 19,985,933 | 19,992,033 | Autoflower | |
| AF locus | | | | |
| QTLl3 | 20,067,897 | 23,470,482 | Terpene Profile | 2 |
| QTLl4 | 40,668,367 | 42,149,848 | Days to Maturity | 2 |
| QTLl5 | 64,562,451 | 79,771,913 | Days to Maturity | 3 |

GWAS revealed the existence of loci involved in agronomic and composition traits (value traits) linked to the autoflower locus on chromosome 1, and where the autoflower allele is in repulsion phase with favorable alleles for these agronomic and composition traits (that is the autoflower allele and unfavorable alleles for agronomic and composition traits are carried by one of the two homologous copies of chromosome 1, while the daylength-sensitive allele and unfavorable alleles for agronomic and composition traits are carried by the other homologous copy of chromosome 1). As a result, autoflower and unfavorable alleles for agronomic and composition traits are generally inherited together. Breaking this undesirable inheritance relationship between autoflower and favorable alleles for agronomic and composition traits requires being able to select very infrequent recombination events that may occur between the autoflower locus and linked loci involved in agronomic and composition traits. Selecting such infrequent recombination events would require the screening of very large numbers of individual plants. Such recombination events are practically impossible to observe phenotypically on individual plants. Therefore, the most and possibly only effective approach to select such desirable recombination events is through the use of the markers located between the autoflower locus and neighboring agronomic and composition trait loci, as illustrated herein.

### Example 15

### QTL Mapping of Autoflower and Agronomic and Composition Traits (Value Traits)

A population of 186 F2 Cannabis sativa plants was generated from a cross between a known photoperiod sensitive (PP) parent and a known photoperiod insensitive/autoflower (AF) parent to conduct a QTL mapping experiment for a number of traits of interest.

Each F2 plant was phenotyped in 2021 for daylength sensitivity (with two phenotypes: PP or AF), CBD content, THC content, and a number of other traits.

Each F2 plant was also genotyped at 600 SNP loci, including one marker very tightly linked to the AF/PP locus on chromosome 1 and fully diagnostic of the daylength sensitivity phenotype (AF marker). A QTL mapping analysis was conducted from the phenotypic and genotypic data, using single-factor analyses of variance (ANOVA), performed with JMP®, Version 16.1.0. SAS Institute Inc., Cary, NC, 1989-2021.

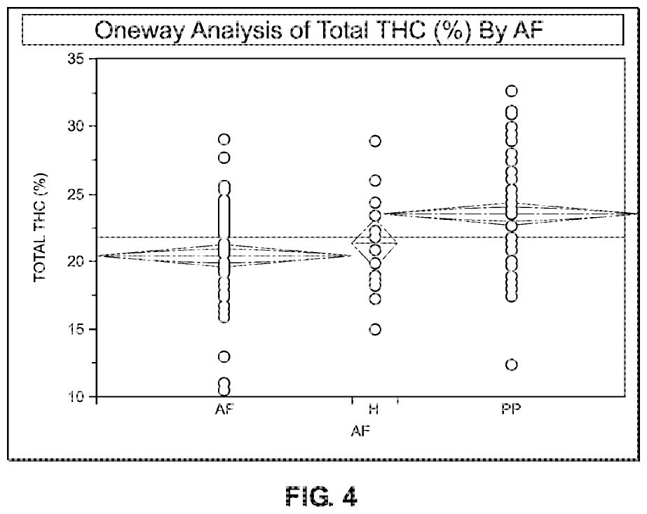

A number of ANOVAs were found to be significant, including that where the dependent variable (phenotype) was THC content (%) and the independent variable (genotype) was the AF marker: (F(2,183)=16.064, p=<0.0001), the allele coming from the AF parent of the cross displaying a significantly lower THC content than the allele coming from the PP parent of that same cross. This evidence of the presence of a THC content QTL in the vicinity of the AF locus, in repulsion with the AF allele (unfavorable THC content allele in coupling with favorable daylength sensitivity allele), contributes to the understanding of the basis for the generally lower performance of AF germplasm when compared to PP germplasm, and sheds light on the fact that some of that difference in performance may be due to unfavorable linkages between AF and other traits, such as THC content as demonstrated here, on chromosome 1. See FIG. 4.

TABLE 9
Summary of Fit

| | |
|---|---|
| Rsquare | 0.149343 |
| Adj Rsquare | 0.140047 |
| Root Mean Square Error | 3.618427 |
| Mean of Response | 21.81034 |
| Observations (or Sum Wgts) | 186 |

Analysis of Variance

| | | Sum of | Mean | | |
|---|---|---|---|---|---|
| Source | DF | Squares | Square | F Ratio | Prob > F |
| AF | 2 | 420.6514 | 210.326 | 16.064 | <.0001 |
| Error | 183 | 2396.022 | 13.093 | | |
| C. Total | 185 | 2816.673 | | | |

Means for One Way
ANOVA

| | | | | Lower | Upper |
|---|---|---|---|---|---|
| Level | Number | Mean | Std Error | 95% | 95% |
| AF | 90 | 20.3805 | 0.38142 | 19.628 | 21.133 |
| H | 16 | 21.3228 | 0.90461 | 19.538 | 23.108 |
| PP | 80 | 23.5164 | 0.40455 | 22.718 | 24.315 |

TABLE 10
Genes linked with autoflower locus on chromosome 1:

| Gene Intervals | Beginning Position (bp) | End Position (bp) |
|---|---|---|
| GI1 | 12,331,257 | 15,023,503 |
| GI2 | 16,178,336 | 19,290,938 |
| GI3 | 19,717,871 | 19,958,734 |
| Exemplary AF locus | 19,985,933 | 19,992,033 |
| GI4 | 19,994,256 | 20,030,132 |
| GI5 | 20,067,897 | 39,266,953 |
| GI6 | 40,668,367 | 60,618,753 |
| GI7 | 64,562,451 | 90,967,989 |

### Example 16

### Evidence for Linkage Between Autoflower Locus and Loci Involved in Agronomic and Composition Traits (Value Traits)

Genes of interest for agronomic and composition traits including Abiotic Stress Response, Autoflower, Defense Response, Flowering, Plant Development and Terpene Synthesis were identified and categorized based on functionality and gene ontology descriptions. The selected genes of interest were placed relative to the markers identified in the AM.

For the sake of simplification genes were grouped into gene intervals. Some of these gene intervals included multiple genes involved in multiple traits. These gene intervals were positioned based on physical position against the Cs10 Genome Assembly (GCA_900626175.2).

### Example 17

### Identification and Use of Markers to Break Unfavorable Associations Between the Autoflower Phenotype and Low Potency—Developmental Leaf-to-Flower Commitment

Based on the evidence for linkage between the autoflower locus and loci involved in agronomic and composition traits, markers are developed to enable the breaking of unfavorable linkage between the autoflower phenotype and the inferior autoflower alleles of other value traits. The use of such markers allows for selection of recombination events between the autoflower locus and other loci involved in other value traits, on chromosome 1, where the autoflower locus is found.

A special focus on potency implicates various kinds of genes that can affect potency, including genes involved in developmental leaf-to-flower commitment. The AF phenotype in Cannabis is often associated with inflorescences that are, on the average, more leafy than most photoperiod varieties. The greater leafiness can contribute to lower potency because (a) trichome density is much lower on leaf tissue than on flower tissue; and (b) cannabinoids are produced and stored in the trichomes. Simply stated, more leaves per flower generally results in fewer trichomes per flower, and therefore a reduced capacity to produce and store cannabinoids.

It is noted that both the AP2 and UPF2 genes are found in the region defined by the markers in Table 3, and that both genes have been functionally characterized to affect flower development and may be involved in the leaf-to-flower commitment during development. Other genes on chromosome 1 that also contribute to leaf-to-flower commitment are also identified, and alleles for these loci are determined in one or more AF plants. These alleles are compared with alleles for the same loci from a variety of Value Phenotype photoperiod plants. Any alleles for floral development genes on chromosome 1, that are different in AF plants as compared with Value Phenotype plants are designated as “AF-associated alleles.”

Having identified AF-associated alleles for genes related to floral development, marker-assisted breeding is conducted using an AF parent and one or more Value Phenotype photoperiod parents. The MAB includes intensive selection against the AF-associated alleles while selecting for presence of an AF allele or, in some cases, selecting for AF phenotype. Progeny plants having an AF allele while having fewer AF-associated alleles than the parent AF plant show increased potency as compared with the AF parent.

### Example 18

### Identification and Use of Markers to Break Unfavorable Associations Between the Autoflower Phenotype and Low Potency—Trichome Size and/or Density

Based on the evidence for linkage between the autoflower locus and loci involved in agronomic and composition traits, markers are developed to enable the breaking of unfavorable linkage between the autoflower phenotype and the inferior autoflower alleles of other value traits. The use of such markers allows for selection of recombination events between the autoflower locus and other loci involved in other value traits, on chromosome 1, where the autoflower locus is found.

A special focus on potency implicates various kinds of genes that can affect potency, including genes involved in trichome size and/or density. Trichome size and/or density have clear implications as to overall potency, because cannabinoids are made and stored in trichomes.

Genes on chromosome 1 that affect trichome size and/or density are identified, and alleles for these loci are determined in one or more AF plants. These alleles are compared with alleles for the same loci from a variety of Value Phenotype photoperiod plants. Any alleles for trichome size/density genes on chromosome 1, that are different in AF plants as compared with Value Phenotype plants are designated as “AF-associated alleles.”

Having identified AF-associated alleles for trichome size/density-related genes, marker-assisted breeding is conducted using an AF parent and one or more Value Phenotype photoperiod parents. The MAB includes intensive selection against the AF-associated alleles while selecting for presence of an AF allele or, in some cases, selecting for AF phenotype. Progeny plants having an AF allele while having fewer AF-associated alleles than the parent AF plant show increased potency as compared with the AF parent.

### Example 19

### Identification and Use of Markers to Break Unfavorable Associations Between the Autoflower Phenotype and Low Potency—THC Biosynthesis

Based on the evidence for linkage between the autoflower locus and loci involved in agronomic and composition traits, markers are developed to enable the breaking of unfavorable linkage between the autoflower phenotype and the inferior autoflower alleles of other value traits. The use of such markers allows for selection of recombination events between the autoflower locus and other loci involved in other value traits, on chromosome 1, where the autoflower locus is found.

A special focus on potency implicates various kinds of genes that can affect potency, including genes involved in THC biosynthesis. THC biosynthesis has clear implications as to overall potency, lower rates of THC biosynthesis will directly affect THC accumulation in floral trichomes.

Genes on chromosome 1 that affect THC biosynthesis are identified, and alleles for these loci are determined in one or more AF plants. These alleles are compared with alleles for the same loci from a variety of Value Phenotype photoperiod plants. Any alleles for THC biosynthesis genes on chromosome 1, that are different in AF plants as compared with Value Phenotype plants are designated as “AF-associated alleles.”

Having identified AF-associated alleles for THC biosynthesis-related genes, marker-assisted breeding is conducted using an AF parent and one or more Value Phenotype photoperiod parents. The MAB includes intensive selection against the AF-associated alleles while selecting for presence of an AF allele or, in some cases, selecting for AF phenotype. Progeny plants having an AF allele while having fewer AF-associated alleles than the parent AF plant show increased potency as compared with the AF parent.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. A method of plant breeding to develop an Autoflower Value Phenotype, comprising
a. providing a first parent plant, having a phenotype defined as a Value Phenotype, wherein the Value Phenotype comprises at least one trait of interest;
b. providing a second parent plant, having an autoflower phenotype;
c. crossing the first and second parent plants;
d. recovering progeny from the crossing step;
e. screening the progeny for presence of at least one autoflower allele using a marker having at least 51% correlation with presence of the autoflower allele;
f. selecting autoflower carrier progeny, wherein cells of said autoflower carrier progeny comprise at least one autoflower allele;
g. conducting further breeding steps using autoflower carrier progeny crossed with plants having the Value Phenotype; and
h. repeating steps e, f, and g until at least one plant having an Autoflower Value Phenotype is obtained.

2. The method of claim 1, wherein the further breeding steps of step f comprise at least one of: a backcross; a self-cross; a sibling cross; and creation of a double haploid.

3. A method of plant breeding to develop a plant with an Autoflower Value Phenotype, comprising
a. providing a first parent plant, having a phenotype defined as a Value Phenotype, wherein the Value Phenotype comprises at least one trait of interest;
b. providing a second parent plant, having an autoflower phenotype;
c. crossing the first and second parent plants;
d. recovering progeny from the crossing step;
e. identifying one or more loci for which the first and second parent plants are polymorphic such that, for each such polymorphic locus, there exists a first-parent allele and a different second-parent allele;
f. screening individuals of the progeny for presence of (1) at least one autoflower allele (2a) presence of one or more first-parent alleles; and/or (2b) absence one or more second-parent alleles, wherein plants meeting criteria (1) and (2) are designed as desirable progeny;
g. selecting the desirable progeny;
h. conducting further breeding steps using the desirable progeny in one or more of subsequent crosses selected from any of (i) a self-cross of a desirable progeny individual; (ii) a cross between different desirable progeny individuals; (iii) a cross between a desirable progeny individual and the first parent plant; and/or (iv) a cross between a desirable progeny individual and a plant having the Value Phenotype that is not the first parent plant; and
i. repeating steps f, g, and h until at least one plant having an Autoflower Value Phenotype is obtained.

4. The method of claim 1, wherein step e employs one or more markers from Table 1.

5. A method of plant breeding to develop an Autoflower Value Phenotype, comprising
a. providing a first parent plant having a phenotype defined as a Value Phenotype, wherein the Value Phenotype comprises at least one trait of interest;
b. providing a second parent plant, having an autoflower phenotype;
c. crossing the first and second parent plants;
d. recovering progeny from the crossing step;
e. screening the progeny phenotypically for presence of at least one autoflower-associated marker and the Value Phenotype;
f. selecting autoflower carrier progeny with the Value Phenotype, wherein cells of said autoflower carrier progeny comprise at least one autoflower-associated marker;
g. conducting further breeding steps using autoflower carrier progeny selfed, sib-mated, or crossed with plants having the Value Phenotype; and
h. repeating steps e, f, and g until at least one plant having an Autoflower Value Phenotype is obtained.

6. (canceled)

7. (canceled)

8. (canceled)

9. The method of claim 1, wherein the modulated day-length sensitivity phenotype is an autoflower phenotype, attenuation of day-length sensitivity, or increase of day-length sensitivity.

10. The method of claim 5, wherein the autoflower-associated marker is selected from Table 1.

11. The method of claim 1, wherein the Value Phenotype comprises at least one trait selected from:
a. high THCA accumulation;
b. specific cannabinoid ratio(s);
c. a composition of terpenes and/or other aromatic molecules;
d. monoecy or dioecy (enable or prevent hermaphroditism);
e. branchless or branched architectures with specific height to branch length ratios or total branch length;
f. high flower to leaf ratios that enable pathogen resilience through improved airflow;
g. high flower to leaf ratios that maximize light penetration and flower development in the vertical canopy space;
h. a finished plant height that enables tractor farming inside high tunnels;
i. a finished plant height and flower to leaf ratio that maximizes light penetration all the way to the ground but minimizes total plant height;
j. trichome size;
k. trichome density;
l. advantageous flower structures for oil or flower production
i. flower diameter length
ii. long or short internodal spacing distance
iii. flower-to-leaf determination ratio (leafiness of flower);
m. metabolites that provide enhanced properties to finished oil products (oxidation resistance, color stability, cannabinoid and terpene stability);
n. specific variants affecting cannabinoid or aromatic molecule biosynthetic pathways;
o. modulators of the flowering time phenotype that increase or decrease maturation time;
p. biomass yield and composition;
q. crude oil yield and composition;
r. resistance to Botrytis, powdery mildew, Fusarium, Pythium, Cladosporium, Alternaria, spider mites, broad mites, russet mites, aphids, nematodes, caterpillars, HLVd or any other Cannabis pathogen or pest of viral, bacterial, fungal, insect, or animal origin; and
s. propensity to host specific beneficial and/or endophytic microflora.

12. The method of claim 3, wherein step e employs one or more markers from Table 1.

13. The method of claim 3, wherein the modulated day-length sensitivity phenotype is an autoflower phenotype, attenuation of day-length sensitivity, or increase of day-length sensitivity.

14. The method of claim 3, wherein the Value Phenotype comprises at least one trait selected from:
a. high THCA accumulation;
b. specific cannabinoid ratio(s);
c. a composition of terpenes and/or other aromatic molecules;
d. monoecy or dioecy (enable or prevent hermaphroditism);
e. branchless or branched architectures with specific height to branch length ratios or total branch length;
f. high flower to leaf ratios that enable pathogen resilience through improved airflow;
g. high flower to leaf ratios that maximize light penetration and flower development in the vertical canopy space;
h. a finished plant height that enables tractor farming inside high tunnels;
i. a finished plant height and flower to leaf ratio that maximizes light penetration all the way to the ground but minimizes total plant height;
j. trichome size;
k. trichome density;
l. advantageous flower structures for oil or flower production
i. flower diameter length
ii. long or short internodal spacing distance
iii. flower-to-leaf determination ratio (leafiness of flower);
m. metabolites that provide enhanced properties to finished oil products (oxidation resistance, color stability, cannabinoid and terpene stability);
n. specific variants affecting cannabinoid or aromatic molecule biosynthetic pathways;
o. modulators of the flowering time phenotype that increase or decrease maturation time;
p. biomass yield and composition;
q. crude oil yield and composition;
r. resistance to Botrytis, powdery mildew, Fusarium, Pythium, Cladosporium, Alternaria, spider mites, broad mites, russet mites, aphids, nematodes, caterpillars, HLVd or any other Cannabis pathogen or pest of viral, bacterial, fungal, insect, or animal origin; and
s. propensity to host specific beneficial and/or endophytic microflora.

16. The method of claim 5, wherein the modulated day-length sensitivity phenotype is an autoflower phenotype, attenuation of day-length sensitivity, or increase of day-length sensitivity.

17. The method of claim 5, wherein the Value Phenotype comprises at least one trait selected from:
a. high THCA accumulation;
b. specific cannabinoid ratio(s);
c. a composition of terpenes and/or other aromatic molecules;
d. monoecy or dioecy (enable or prevent hermaphroditism);
e. branchless or branched architectures with specific height to branch length ratios or total branch length;
f. high flower to leaf ratios that enable pathogen resilience through improved airflow;
g. high flower to leaf ratios that maximize light penetration and flower development in the vertical canopy space;
h. a finished plant height that enables tractor farming inside high tunnels;
i. a finished plant height and flower to leaf ratio that maximizes light penetration all the way to the ground but minimizes total plant height;
j. trichome size;
k. trichome density;
l. advantageous flower structures for oil or flower production
i. flower diameter length
ii. long or short internodal spacing distance
iii. flower-to-leaf determination ratio (leafiness of flower);
m. metabolites that provide enhanced properties to finished oil products (oxidation resistance, color stability, cannabinoid and terpene stability);
n. specific variants affecting cannabinoid or aromatic molecule biosynthetic pathways;
o. modulators of the flowering time phenotype that increase or decrease maturation time;
p. biomass yield and composition;
q. crude oil yield and composition;
r. resistance to Botrytis, powdery mildew, Fusarium, Pythium, Cladosporium, Alternaria, spider mites, broad mites, russet mites, aphids, nematodes, caterpillars, HLVd or any other Cannabis pathogen or pest of viral, bacterial, fungal, insect, or animal origin; and
s. propensity to host specific beneficial and/or endophytic microflora.
